(19) United States
(12) Patent Application Publication
(10) Pub. No.: US 2024/0277983 A1
(43) Pub. Date: Aug. 22, 2024

(54) SYSTEMS FOR DELIVERING DEVICES FOR REGULATING BLOOD PRESSURE ACROSS AN ATRIAL SEPTUM

(71) Applicant: V-Wave Ltd., Caesarea (IL)

(72) Inventors: Nir NAE, Binyamina (IL); Michael RABINER, Hadera (IL); James S. WHITING, Los Angeles, CA (US)

(73) Assignee: V-Wave Ltd., Caesarea (IL)

(21) Appl. No.: 18/651,630

(22) Filed: Apr. 30, 2024

Related U.S. Application Data
(63) Continuation of application No. PCT/IB2022/060621, filed on Nov. 3, 2022.
(60) Provisional application No. 63/263,535, filed on Nov. 4, 2021.

(51) Int. Cl. A61M 27/00; A61M 25/10

(52) U.S. Cl. CPC A61M 27/002; A61F 2/82; A61F 2/844; A61F 2/86; A61F 2/95; A61F 2/9522; A61F 2/958; A61M 25/10; A61F 2002/821

(57) ABSTRACT

Systems and methods are provided for delivering an interatrial shunt device to a puncture of the atrial septum of a patient. The delivery devices described herein do not require a separate dilator as the components thereof function as a dilator for enlarging the puncture of the atrial septum prior to delivery of the interatrial shunt. For example, the integrated dilator may include an expandable portion that transitions to a contracted state after dilation of the puncture for deployment of the shunt. Alternatively, the delivery sheath may have an expandable portion that forms part of the dilator, such that the sheath may be expanded to permit deployment of the shunt. For example, a balloon catheter may be used to expand the sheath. In another embodiment, a portion of the shunt device may form part of the dilator, prior to deployment of the shunt.

### CROSS-REFERENCE TO RELATED APPLICATION

This application claims priority to U.S. Provisional Patent Appl. No. 63/263,535, filed Nov. 4, 2021, the entire contents of which are incorporated herein by reference.

### FIELD OF USE

This application generally relates to devices and methods for delivering implantable devices to the atrial septum, particularly in subjects with heart pathologies such as pulmonary arterial hypertension (PAH), congestive heart failure (CHF) or myocardial infarction (MI).

### BACKGROUND

Pulmonary arterial hypertension (PAH) occurs when the pressure within the blood vessels and lungs becomes too high. PAH may be caused by obstruction in the arteries in the lung such as the development of scar tissue in the blood vessels of the lungs, but in many cases, the cause is unknown. Under normal conditions, the pressure within the right side of the heart and the blood vessels of the lungs is lower than the rest of the body which maximizes oxygenation of the blood in the lungs. With PAH, the heart must work harder under greater pressure to pump blood through the arteries in the lungs, weakening the heart muscles over time. As a result, the heart may be unable to sufficiently pump blood to the lungs to be oxygenated to keep the body functioning normally.

Heart failure is the physiological state in which cardiac output is insufficient to meet the needs of the body or to do so only at a higher filling pressure. There are many underlying causes of HF, including myocardial infarction, coronary artery disease, valvular disease, hypertension, and myocarditis. Chronic heart failure is associated with neurohormonal activation and alterations in autonomic control. Although these compensatory neurohormonal mechanisms provide valuable support for the heart under normal physiological circumstances, they also play a fundamental role in the development and subsequent progression of HF.

For example, one of the body's main compensatory mechanisms for reduced blood flow in HF is to increase the amount of salt and water retained by the kidneys. Retaining salt and water, instead of excreting it via urine, increases the volume of blood in the bloodstream and helps to maintain blood pressure. However, the larger volumes of blood also cause the heart muscle, particularly the ventricles, to become enlarged. As the heart chambers become enlarged, the wall thickness decreases and the heart's contractions weaken, causing a downward spiral in cardiac function. Another compensatory mechanism is vasoconstriction of the arterial system, which raises the blood pressure to help maintain adequate perfusion, thus increasing the load that the heart must pump against.

In low ejection fraction (EF) heart failure, high pressures in the heart result from the body's attempt to maintain the high pressures needed for adequate peripheral perfusion. However, as the heart weakens as a result of such high pressures, the disorder becomes exacerbated. Pressure in the left atrium may exceed 25 mmHg, at which stage fluids from the blood flowing through the pulmonary circulatory system transudate or flow out of the pulmonary capillaries into the pulmonary interstitial spaces and into the alveoli, causing lung congestion and, if untreated, the syndrome of acute pulmonary edema and death.

Table 1 lists typical ranges of right atrial pressure (RAP), right ventricular pressure (RVP), left atrial pressure (LAP), left ventricular pressure (LVP), cardiac output (CO), and stroke volume (SV) for a normal heart and for a heart suffering from HF. In a normal heart beating at around 70 beats/minute, the stroke volume needed to maintain normal cardiac output is about 60 to 100 milliliters. When the preload, after-load, and contractility of the heart are normal, the pressures required to achieve normal cardiac output are listed in Table 1. In a heart suffering from HF, the hemodynamic parameters change (as shown in Table 1) to maintain peripheral perfusion.

TABLE 1

| Parameter | Normal Range | HF Range |
|---|---|---|
| RAP (mmHg) | 2-6 | 6-20 |
| RVSP (mmHg) | 15-25 | 20-80 |
| LAP (mmHg) | 6-12 | 15-50 |
| LVEDP (mmHg) | 6-12 | 15-50 |
| CO (liters/minute) | 4-8 | 2-6 |
| SV (milliliters/beat) | 60-100 | 30-80 |

HF is generally classified as either systolic heart failure (SHF) or diastolic heart failure (DHF). In SHF, the pumping action of the heart is reduced or weakened. A common clinical measurement is the ejection fraction, which is the volume of blood ejected out of the left ventricle (stroke volume) divided by the maximum volume in the left ventricle at the end of diastole or relaxation phase. A normal ejection fraction is greater than 50%. Systolic heart failure generally causes a decreased ejection fraction of less than 40%. Such patients have heart failure with reduced ejection fraction (HFrEF). A patient with HFrEF may usually have a larger left ventricle because of a phenomenon called “cardiac remodeling” that occurs secondary to the higher ventricular pressures.

In DHF, the heart generally contracts normally, with a normal ejection fraction, but is stiffer, or less compliant, than a healthy heart would be when relaxing and filling with blood. Such patients are said to have heart failure with preserved ejection fraction (HFpEF). This stiffness may impede blood from filling the heart and produce backup into the lungs, which may result in pulmonary venous hypertension and lung edema. HFpEF is more common in patients older than 75 years, especially in women with high blood pressure.

Both variants of HF have been treated using pharmacological approaches, which typically involve the use of vasodilators for reducing the workload of the heart by reducing systemic vascular resistance, as well as diuretics, which inhibit fluid accumulation and edema formation, and reduce cardiac filling pressure. No pharmacological therapies have been shown to improve morbidity or mortality in HFpEF whereas several classes of drugs have made an important impact on the management of patients with HFrEF, including renin-angiotensin antagonists, beta blockers, and mineralocorticoid antagonists. Nonetheless, in general, HF remains a progressive disease and most patients have deteriorating cardiac function and symptoms over time. In the U.S., there are over 1 million hospitalizations annually for acutely worsening HF and mortality is higher than for most forms of cancer.

In more severe cases of HFrEF, assist devices such as mechanical pumps are used to reduce the load on the heart by performing all or part of the pumping function normally done by the heart. Chronic left ventricular assist devices (LVAD), and cardiac transplantation, often are used as measures of last resort. However, such assist devices typically are intended to improve the pumping capacity of the heart, to increase cardiac output to levels compatible with normal life, and to sustain the patient until a donor heart for transplantation becomes available. Such mechanical devices enable propulsion of significant volumes of blood (liters/min), but are limited by a need for a power supply, relatively large pumps, and pose a risk of hemolysis, thrombus formation, and infection. Temporary assist devices, intra-aortic balloons, and pacing devices have also been used.

Various devices have been developed using stents to modify blood pressure and flow within a given vessel, or between chambers of the heart. Implantable interatrial shunt devices have been successfully used in patients with severe symptomatic heart failure. By diverting or shunting blood from the left atrium (LA) to the right atrium (RA), the pressure in the LA is lowered or prevented from elevating as high as it would otherwise (left atrial decompression). Such an accomplishment would be expected to prevent, relieve, or limit the symptoms, signs, and syndromes associated with pulmonary congestion. These include severe shortness of breath, pulmonary edema, hypoxia, the need for acute hospitalization, mechanical ventilation, and death.

Percutaneous implantation of interatrial shunts generally requires transseptal catheterization immediately preceding shunt device insertion. The transseptal catheterization system is placed from an entrance site in the femoral vein, across the interatrial septum in the region of fossa ovalis (FO), which is the central and thinnest region of the interatrial septum. This is the same general location where a congenital secundum atrial septal defect (ASD) would be located. The FO in adults is typically 15-20 mm in its major axis dimension and ≤3 mm in thickness, but in certain circumstances may be up to 10 mm thick. LA chamber access may be achieved using a host of different techniques familiar to those skilled in the art, including but not limited to: needle puncture, stylet puncture, screw needle puncture, and radiofrequency ablation. The passageway between the two atria is dilated to facilitate passage of a shunt device having a desired orifice size. Dilation generally is accomplished by advancing a tapered sheath/dilator catheter system or inflation of an angioplasty type balloon across the FO. A limitation of advancing a typical separate tapered dilator is that after dilating the septum, the dilator must be removed from the sheath before any device to be delivered can be loaded into the sheath and advanced for deployment.

Moreover, devices such as those described in U.S. Pat. No. 5,312,341 to Turi, have been theorized for transseptal catheterization. Specifically, these devices have a retaining means such as an inflatable balloon that is inflated within the left atrium of the patient to prevent inadvertent retraction of the distal tip of the sheath from the left atrium during subsequent portions of the catheterization procedure.

In view of the foregoing, it would be desirable to provide devices for delivering implantable devices to the atrial septum of the heart to reduce left atrial pressure, while reducing the number of delivery tools required.

It would further be desirable to provide devices and methods for controlled positioning and delivery of atrial shunt devices.

### SUMMARY

The present disclosure overcomes the drawbacks of previously known systems and methods by providing systems and methods for delivering a shunt to an atrial septum of a patient. For example, the apparatus may include a sheath having a proximal region, a distal region, and a sheath lumen extending therethrough, the sheath lumen sized and shaped to receive the shunt in a collapsed delivery state, and a balloon catheter configured to be moveably disposed within the sheath lumen. The balloon catheter may include a balloon configured to transition between a deflated collapsed state and an inflated expanded state adjacent to the distal region to form a continuous, step-free transition between the balloon and the distal region of the sheath. The apparatus further may include a handle having one or more actuators configured to be actuated to deploy the shunt at the atrial septum.

In addition, the apparatus may include a pusher slidably disposed within the sheath lumen. The pusher may be operatively coupled to a pusher actuator of the one or more actuators of the handle, such that the pusher actuator may be configured to be actuated to move the pusher within the sheath lumen. For example, the pusher actuator may be configured to be actuated to move the pusher distally relative to the sheath, such that a distal end of the pusher engages with a proximal portion of the shunt in the collapsed delivery state to thereby move the shunt distally relative to the sheath until a distal portion of the shunt is exposed beyond the distal region of the sheath and transitions from the collapsed delivery state to an expanded deployed state.

The apparatus further may include a release knot slidably disposed within the sheath lumen, the release knot configured to be releasably engaged with the shunt, e.g., at a proximal portion of the shunt, via a hitch knot, e.g., a painters hitch knot or a Quick Tie and Release (QTaR) hitch knot. For example, a first end of the release knot may be operatively coupled to a release actuator of the one or more actuators of the handle and a second end of the release knot may be operatively coupled to a retrieval actuator of the one or more actuators of the handle, such that actuation of the release actuator causes the hitch knot to disassemble and disengage the release knot from the shunt, and actuation of the retrieval actuator causes retraction of the shunt proximally within the sheath lumen via the hitch knot for halfway retrieval of the shunt. Alternatively, a first end of the release knot may be operatively coupled to a release actuator of the one or more actuators of the handle and a second end of the release knot may be operatively coupled to a distal portion of the pusher, such that actuation of the release actuator causes the hitch knot to disassemble and disengage the release knot from the shunt, and actuation of the pusher actuator causes retraction of the shunt proximally within the sheath lumen via the hitch knot for halfway retrieval of the shunt.

Moreover, the balloon may be configured to be deflated to permit deployment of the shunt through the distal region of the sheath. The balloon catheter may include a fluid lumen configured to fluidically couple the balloon and a fluid source. In addition, the balloon catheter may be operatively coupled to a balloon catheter actuator of the one or more actuators of the handle, such that actuation of the balloon catheter actuator causes the balloon catheter to move relative to the sheath.

In accordance with another aspect of the present disclosure, a method for delivering a shunt to an atrial septum of a patient is provided. The method may include inflating a balloon adjacent to a distal region of a sheath to form a continuous, step-free transition between the balloon and the distal region of the sheath, the balloon disposed on a distal portion of a balloon catheter slidably disposed within a lumen of the sheath; delivering the inflated balloon and the sheath through an opening of the atrial septum, such that the inflated balloon and the sheath dilates the opening of the atrial septum; deflating the balloon; advancing the shunt distally within the lumen of the sheath in a collapsed delivery state until a distal portion of the shunt is exposed beyond the distal region of the sheath and transitions to an expanded deployed state within a first atrium; and retracting the sheath proximally relative to the atrial septum until a proximal portion of the shunt is exposed beyond the distal region of the sheath and transitions to the expanded deployed state within a second atrium, such that the shunt is deployed at the atrial septum.

The shunt may releasably engaged with a release knot, such that pulling a first end of the release knot causes the release knot to disengage from the shunt, and pulling a second end of the release knot causes retraction of the shunt proximally within the lumen of the sheath for halfway retrieval of the shunt. For example, the release knot may be releasably engaged with the shunt via a painters hitch knot or a Quick Tie and Release (QTaR) hitch knot.

Accordingly, prior to retracting the sheath proximally relative to the atrial septum until the proximal portion of the shunt is exposed beyond the distal region of the sheath, the method further may include pulling the first end of the release knot to disengage the release knot from the shunt. Moreover, prior to retracting the sheath proximally relative to the atrial septum until the proximal portion of the shunt is exposed beyond the distal region of the sheath, the method further may include pulling the second end of the release knot to transition the distal portion of the shunt to the collapsed delivery state within the lumen of the sheath for halfway retrieval of the shunt. In addition, prior to retracting the sheath proximally relative to the atrial septum until the proximal portion of the shunt is exposed beyond the distal region of the sheath, the method further may include retracting the sheath and the shunt proximally relative to the atrial septum until the distal portion of the shunt contacts the atrial septum from within the first atrium.

Advancing the shunt distally within the lumen of the sheath may include advancing a pusher distally within the lumen of the sheath, such that a distal end of the pusher engages with the proximal portion of the shunt in the collapsed delivery state within the lumen of the sheath. Accordingly, prior to retracting the sheath proximally relative to the atrial septum until the proximal portion of the shunt is exposed beyond the distal region of the sheath, the method further may include retracting the shunt proximally within the lumen of the sheath to transition the distal portion of the shunt to the collapsed delivery state within the lumen of the sheath for halfway retrieval of the shunt. In some embodiments, prior to retracting the shunt proximally within the lumen of the sheath, the method may include retracting the pusher distally relative to the sheath. The method further may include retracting the balloon catheter and the deflated balloon proximally within the lumen of the sheath to a position proximal to the shunt in the collapsed delivery state prior to advancing the shunt within the lumen of the sheath. In addition, the method may include removing the sheath and the balloon catheter from the patient.

In accordance with another aspect of the present disclosure, another apparatus for delivering a shunt to an atrial septum of a patient is provided. The apparatus may include a sheath configured to be advanced through a hole in the atrial septum, the sheath having a proximal region, a distal region, and a sheath lumen extending therethrough, the sheath lumen sized and shaped to receive the shunt in a collapsed delivery state, and a dilator moveably disposed within the sheath lumen. The dilator may include an expandable portion configured to transition between a first state and a second state where the expandable portion engages with the distal region of the sheath. Accordingly, the dilator and the sheath may be configured to dilate the hole in the atrial septum as tissue surrounding the hole is smoothly guided over the distal portion of the dilator and the sheath as the apparatus is advanced through hole in the atrial septum. The dilator may include a guidewire lumen sized and shaped to receive a guidewire.

In accordance with one aspect of the present disclosure, the dilator further may include a dilation catheter moveably disposed within the sheath lumen, and a cone-shaped tip coupled to a distal end of the dilation catheter, and to a distal portion of the expandable portion of the dilator. For example, in the first state, the proximal portion of the expandable portion may be contracted radially inward toward the dilation catheter, and, in the second state, a proximal portion of the expandable portion may be removeably engaged with the distal region of the sheath to form a continuous, step-free transition between the sheath and the expandable portion of the dilator. The dilation catheter may be configured to be moved distally relative to the sheath to cause the expandable portion of the dilator to transition from the second state to the first state. Moreover, the expandable portion of the dilator may be biased toward the first state.

In the second state, the distal portion of the expandable portion of the dilator may engage with an outer surface of the distal region of the sheath. Accordingly, the apparatus may have a continuous, step-free transition between the sheath and the expandable portion of the dilator when the expandable portion is in the second state. The guidewire lumen may extend through the cone-shaped tip and the dilation catheter. In addition, the sheath may be configured to be moved proximally relative to the dilator when the expandable portion of the dilator is in the first state to deploy the shunt at the atrial septum. The apparatus further may include a hollow catheter, e.g., a PEEK tube, moveably disposed within the sheath lumen, the hollow catheter sized and shaped to receive the dilation catheter. For example, in the first state, the proximal portion of the expandable portion of the dilator may be disposed within a distal region of the hollow catheter.

In accordance with another aspect of the present disclosure, the distal region of the sheath may be configured to transition between a contracted state and an expanded state, and the expandable portion of the dilator may include a proximal portion coupled to an outer tube moveably disposed within the sheath lumen, and a cone-shaped distal portion coupled to an inner tube moveably disposed within the outer tube such that the cone-shaped distal portion is moveable relative to the proximal portion between the second state where the distal region of the sheath is sandwiched between the proximal portion and the cone-shaped distal portion in the contracted state, and the first state where the distal region of the sheath disengages with the proximal portion and the cone-shaped distal portion to transition to the expanded state. Accordingly, the apparatus may have a continuous, step-free transition between the cone-shaped distal portion and the distal region of the sheath when the distal region of the sheath is in the contracted state.

In the contracted state, the distal region of the sheath may have a plurality of longitudinal slits disposed circumferentially along the distal region, and extending from a distal end of the distal region toward the proximal region of the sheath. Moreover, in the expanded state, the distal region of the sheath may be expanded along the plurality of longitudinal slits such that each of the plurality of longitudinal slits comprises a V-shape. The distal region of the sheath may be biased toward the expanded state. Moreover, the distal region of the sheath may include an elastic material encapsulated with a biocompatible material. For example, the elastic material may be superelastic Nitinol, and the biocompatible material may be a polyether block amide. The guidewire lumen may extend through the cone-shaped distal portion and the inner tube.

In accordance with another aspect of the present disclosure, the expandable portion of the dilator may include an expandable braided tip coupled to an inner tube moveably disposed within the sheath lumen, the expandable braided tip configured to transition between the first state and the second state. Moreover, a distal portion of the shunt may be configured to transition between the collapsed delivery state where the distal portion of the shunt forms a continuous, step-free transition between the distal portion of the shunt and the expandable braided tip when the expandable braided tip is in the second state, and an expanded deployed state. A proximal end of the expandable braided tip may be coupled to an outer tube and a distal end of the expandable braided tip may be coupled to the inner tube, and the inner tube may be moveably disposed within a lumen of the outer tube, such that the proximal end of the expandable braided tip is moveable relative to the distal end of the expandable braided tip to transition the expandable braided tip between the first state and the second state. In addition, the distal portion of the shunt may be configured to transition from the collapsed delivery state to the expanded deployed state upon application of heat. Accordingly, the sheath further may include a fluid lumen configured to deliver heated liquid to the distal portion of the shunt. The apparatus may have a continuous, step-free transition between the distal portion of the shunt and the distal region of the sheath when the distal portion of the shunt is in the collapsed delivery state. The guidewire lumen may extend through the inner tube.

In accordance with another aspect of the present disclosure, the distal region of the sheath may be configured to transition between a contracted state and an expanded state, and the expandable portion of the dilator may include a balloon coupled to balloon catheter configured to be moveably disposed within the sheath lumen. The balloon may be configured to be inflated from the first state to the second state to transition the distal region from the contracted state to the expanded state. For example, in the contracted state, the distal region of the sheath may define an opening and may have a plurality of longitudinal slits disposed circumferentially along the distal region, and extending from the opening toward the proximal region of the sheath, and in the expanded state, the distal region of the sheath may be expanded along the plurality of longitudinal slits such that each of the plurality of longitudinal slits comprises a V-shape. A distal tip of the balloon may be configured to extend through the opening to form a continuous, step-free transition between the balloon and the distal region of the sheath. In addition, the plurality of longitudinal slits may define a plurality of fingers of the distal region, and a distal end of each of the plurality of fingers may have a round shape. Moreover, the distal region of the sheath may include a shape-memory material configured to cause the distal region to return to the contracted state upon exposure to heat.

In accordance with another aspect of the present disclosure, the expandable portion of the dilator may include a balloon coupled to a balloon catheter configured to be moveably disposed within the sheath lumen. The balloon may be configured to transition between the first state and the second state adjacent to the distal region to form a continuous, step-free transition between the balloon and the distal region of the sheath. The apparatus further may include a pusher slidably disposed within the sheath lumen. The pusher may have a pusher lumen sized and shaped to slidably receive the balloon catheter therethrough, and a distal end configured to engage with a proximal portion of the shunt in the collapsed delivery state to thereby move the shunt distally relative to the sheath. The apparatus further may include a release knot slidably disposed within the sheath lumen. For example, the release knot may be configured to be releasably engaged with the shunt via a hitch knot, e.g., a painters hitch knot or a Quick Tie and Release (QTaR) hitch knot, such that pulling a first end of the release knot causes the release knot to disengage from the shunt, and pulling a second end of the release knot causes retraction of the shunt proximally within the sheath lumen for halfway retrieval of the shunt.

In some embodiments, the release knot may be configured to be releasably engaged with a proximal portion of the shunt. Additionally, the first and second ends of the release knot may pass through a central passageway of the shunt toward a middle portion of the shunt, and loop around an outer surface of the middle portion of the shunt and back towards the hitch knot, such that pulling the second end of the release knot causes the shunt to transition toward the collapsed delivery state. Alternatively, the release knot may be configured to be releasably engaged with a middle portion of the shunt, and the first and second ends of the release knot may loop around an outer surface of the middle portion of the shunt, such that pulling the second end of the release knot causes the shunt to transition toward the collapsed delivery state. The balloon may be configured to be deflated to permit deployment of the shunt through the distal region of the sheath. Moreover, the balloon catheter may have a fluid lumen configured to fluidically couple the balloon and a fluid source.

### BRIEF DESCRIPTION OF THE DRAWINGS

FIGS. 1A to 1D illustrate an exemplary device for delivering an interatrial shunt device to the atrial septum in accordance with the present disclosure.

FIGS. 2A to 2F illustrate an alternative exemplary device for delivering an interatrial shunt device to the atrial septum in accordance with the present disclosure.

FIGS. 3A to 3G illustrate another alternative exemplary device for delivering an interatrial shunt device to the atrial septum in accordance with the present disclosure.

FIGS. 4A and 4B illustrate yet another alternative exemplary device for delivering an interatrial shunt device to the atrial septum in accordance with the present disclosure.

FIG. 5A illustrates yet another alternative exemplary device for delivering an interatrial shunt device to the atrial septum in accordance with the present disclosure.

FIG. 5B illustrates an exemplary handle for actuating the delivery device of FIG. 5A, constructed in accordance with the principles of the present disclosure.

FIG. 6A illustrates an exemplary knot mechanism of the delivery device of FIG. 5A.

FIG. 6B illustrates an alternative exemplary knot mechanism in accordance with the principles of the present disclosure.

FIGS. 7A to 7H illustrate exemplary method steps for delivering an interatrial shunt device to the atrial septum using the delivery device of FIGS. 5A and 5B in accordance with the present disclosure.

FIGS. 7I to 7K illustrate exemplary method steps for half-way retrieval of the interatrial shunt device using the delivery device of FIGS. 5A and 5B in accordance with the present disclosure.

FIGS. 8A to 8G illustrate exemplary method steps for delivering an interatrial shunt device to the atrial septum using another exemplary delivery device in accordance with the present disclosure.

FIGS. 8H and 8I illustrate exemplary method steps for half-way retrieval of the interatrial shunt device using the delivery device of FIGS. 8A to 8G in accordance with the present disclosure.

### DETAILED DESCRIPTION

Embodiments of the present invention are directed to devices for delivering implantable devices to a wall of the heart such as the atrial septum, and thus may be useful in treating subjects suffering from heart failure, myocardial infarction, pulmonary hypertension, or other disorders associated with elevated atrial pressure. For example, the inventive device may be designed to deliver an hourglass or “diabolo” shaped shunt device, preferably formed of a shape memory metal as described in U.S. Pat. No. 9,629,715 to Nitzan, U.S. Pat. No. 10,076,403 to Eigler, and U.S. Pat. No. 11,458,287 to Eigler, each assigned to the assignee of the present invention, the entire contents of each of which are incorporated herein by reference. The delivery devices described herein are configured to lodge the shunt securely in a hole in a heart wall such as the atrial septum, preferably the fossa ovalis, to function as an interatrial shunt, allowing blood flow between the left atrium and the right atrium.

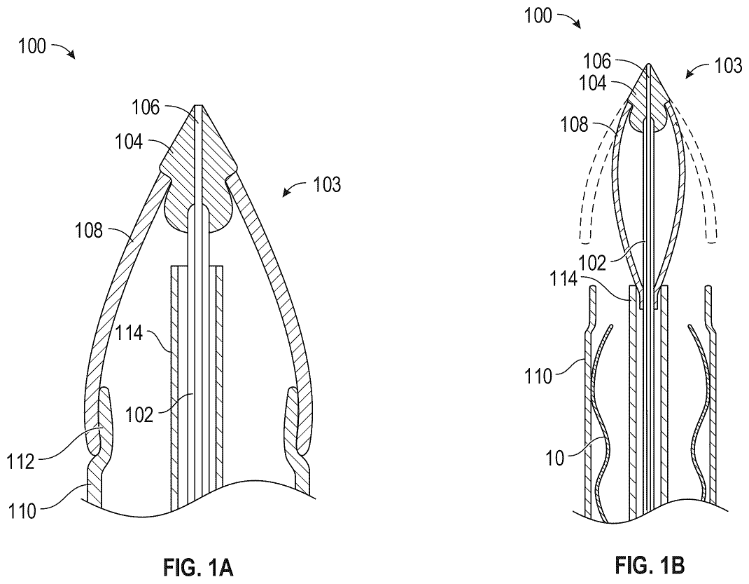

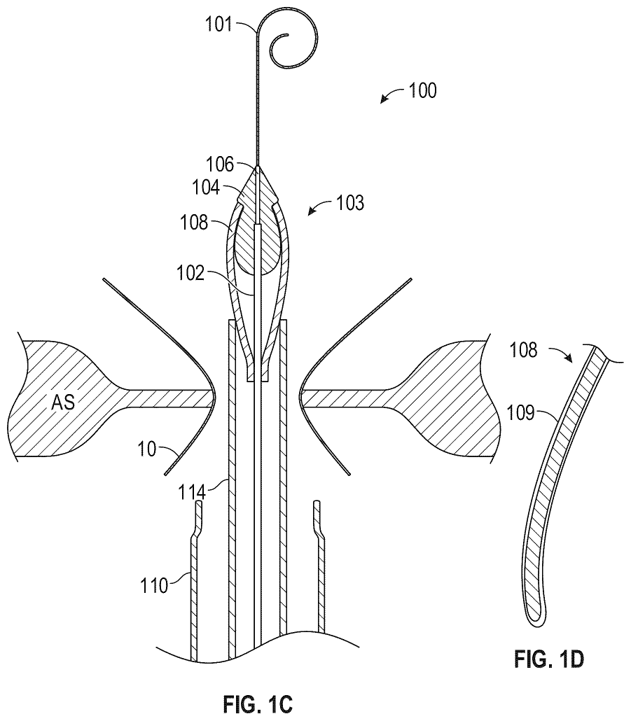

Referring now to FIGS. 1A to 1D, exemplary delivery device 100 for delivering interatrial shunt device 10 to the atrial septum is provided. As shown in FIG. 1A, delivery device 100 includes sheath 110 removeably coupled to dilator 103. Sheath 110 has a lumen extending from distal region 112 of sheath 110 to the proximal region of the sheath external to the patient. The lumen of sheath 110 is sized and shaped to receive shunt 10 in its collapsed delivery state. As shown in FIG. 1A, distal region 112 of sheath 110 may have an outer diameter that is less than the outer diameter of rest of the length of sheath 110 extending from distal region 112 toward the proximal region of sheath 110.

Dilator 103 includes dilation catheter 102 moveably disposed within the lumen of sheath 110, such that dilation catheter 102 may be moved relative to sheath 110, e.g., via actuation of a handle external to the patient that is independently coupled to sheath 110 and dilation catheter 102. In addition, dilator 103 includes dilator tip 104 coupled to the distal end of dilation catheter 102. Dilator tip 104 may be a low durometer soft tip and may have an atraumatic cone shape that may be inserted through a puncture of the atrial septum to enlarge the puncture without damaging the surrounding tissue. Alternatively, dilator tip 104 may have a sharp needle tip that may be used to create the puncture within the atrial septum, such that further advancement of dilator tip 104 across the atrial septum enlarges the puncture.

Dilator 103 may have a guidewire lumen 106 sized and shaped to receive a guidewire therethrough, such that device 100 may be advanced over a conventional guidewire across the atrial septum. Accordingly, guidewire lumen 106 may extend through dilator tip 104 and dilation catheter 102. Moreover, dilator 103 may include expandable portion 108. Expandable portion 108 may be coupled at its distal portion to dilator tip 104, and extend toward sheath 110. In one embodiment, dilator tip 104 and expandable portion 108 are formed of a unitary construction. As shown in FIG. 1D, at least expandable portion 108 of dilator 103 may be encapsulated with biocompatible material 109, e.g., polyether block amide (PEBA), such as PEBAX® (made available by Arkema, Colombes, France).

Expandable portion 108 may be formed of an elastic material, e.g., superelastic Nitinol, and may be transitionable between an expanded state and a contracted state. For example, expandable portion 108 may be heat-set during manufacturing in the contracted state, such that expandable portion 108 is biased toward the contracted state. Accordingly, prior to insertion into the patient, expandable portion 108 may be expanded and fit over distal region 112 of sheath 110. Specifically, as shown in FIG. 1A, in the expanded state, the proximal portion of expandable portion 108 may be engaged with the outer surface of distal region 112, to thereby form a step-free transition between sheath 110 and expandable portion 108 of dilator 103 when expandable portion 108 is in the expanded state. Thus, when the proximal portion of expandable portion 108 is engaged with the outer surface of distal region 112 of sheath 110, the proximal portion of expandable portion 108 has the same outer diameter as the portion of sheath 110 adjacent to distal region 112. The step-free transition between sheath 110 and expandable portion 108 results from distal region 112 having an outer diameter that is less than the outer diameter of the rest of sheath 110. Although FIG. 1A illustrates sheath 110 having a constant thickness along its longitudinal length, e.g., including distal region 112, and thus having an inner diameter varying from sheath 110 to distal region 112, alternatively, sheath 110 and distal region 112 may have a constant inner diameter along its longitudinal length.

As shown in FIG. 1A, device 100 may include hollow catheter 114, e.g., PEEK tube, moveably disposed within the lumen of sheath 110. Hollow catheter 114 may be sized and shaped to receive dilation catheter 102, and at least a portion of the proximal portion of expandable portion 108 therethrough. Accordingly, dilation catheter 102, hollow catheter 114, shunt 10 in its collapsed delivery state, and sheath 110 may be concentric. Dilation catheter 102, hollow catheter 114, and sheath 110 may each be coupled at their proximal regions to a handle for use by a clinician, such that each component may be independently actuated via the handle.

As shown in FIG. 1B, upon movement of dilator 103 distally relative to sheath 110, expandable portion 108 of dilator 103 will disengage with distal region 112 of sheath 110 and return to its contracted state, as distal region 112 will no longer exert a radially outward force on the inner surface of the proximal portion of expandable portion 108. Accordingly, expandable portion 108 will be contracted radially inward toward dilation catheter 102. In its contracted state, the proximal portion of expandable portion 108 may be adjacent to the opening into hollow catheter 114. Accordingly, upon retraction of dilation catheter 102 relative to hollow catheter 114, at least a portion of the proximal portion of expandable portion 108 may be received by the distal region of hollow catheter 114, thereby causing expandable portion 108 to contract even further. Shunt 10 may be deployed by retracting sheath 110 proximally relative to hollow catheter 114 and dilator 103, as shown in FIG. 1C, to thereby implant shunt 10 at atrial septum AS.

Loading of shunt 10 into delivery device 100, e.g., during manufacturing or in a preparatory step, may proceed as follows. First, hollow catheter 114 may be advanced over dilation catheter 102 until the distal region of hollow tube 114 is adjacent to expandable portion 108 of dilator 103. Hollow catheter 114 may be further advanced over at least a portion of the proximal portion of expandable portion 108 to receive the proximal portion of expandable portion 108 therein, providing rigidity to the combined structure of hollow catheter 114 and dilator 103. Hollow catheter 114 and dilator 103 together may be advanced distally through the lumen of sheath 110 until at least dilator 103 is exposed beyond proximal region 112 of sheath 110, such that dilation catheter 102, hollow catheter 114, shunt 10 in its collapsed delivery state, and sheath 110 are concentric. Alternatively, the combined structure of hollow catheter 114 and dilator 103 may be back-loaded through the distal opening of sheath 110 until dilator 103 is adjacent to distal region 112.

Next, dilation catheter 102 may be moved distally relative to hollow catheter 114, such that expandable portion 108 is no longer within hollow catheter 114 and may be expanded radially outwardly away from dilation catheter 102 and positioned over the outer surface of distal region 112 of sheath 110. Upon release of expandable portion 108 over distal region 112, distal region 112 will maintain expandable portion 108 in its expanded state, such that the proximal portion of expandable portion 108 is fitted onto distal region 112. Accordingly, sheath 110, expandable portion 108, and tip 104 form a smooth and continuous dilator assembly, as shown in FIG. 1A, suitable for inserting into a blood vessel over a guidewire and advancing across the interatrial septum.

Next, shunt 10 may be collapsed to its collapsed delivery state within sheath 110, for example, using tools as described in U.S. Pat. No. 9,713,696 to Yacoby or U.S. Patent App. Pub. No. 2020/0315599 to Nac, each assigned to the assignee of the present invention, the entire contents of each of which are incorporated by reference herein. Delivery device 100 is then ready to deliver shunt 10. Delivery of shunt 10 using delivery device 100 described above may proceed as follows. Guidewire 101 may be advanced to the target location through a puncture of the atrial septum, e.g., within the left atrium of the patient. Device 100 may be advanced over guidewire 101 via guidewire lumen 106 until dilator tip 104 comes into contact with the puncture of the atrial septum. Device 100 may be further advanced such that dilator 103 enlarges the puncture of the atrial septum as the tissue surrounding the puncture is smoothly guided over dilator tip 104, followed by expandable portion 108, and sheath 110. Unlike other known delivery systems, a separate dilator is not required to enlarge the puncture of the atrial septum, which must be subsequently removed prior to loading shunt into sheath 110.

Under visualization methods such as fluoroscopy and/or ultrasound imaging such as trans-esophageal echo (TEE) or intracardiac echo (ICE), the target position of device 100 relative to the atrial septum may be verified, e.g., via a radiopaque marker on sheath 110. Next, dilation catheter 102 may be moved distally relative to sheath 110, thereby causing expandable portion 108 to disengage from distal region 112 and transition from its expanded state to its contracted state toward dilation catheter 102, e.g., by virtue of its superelasticity, as shown in FIG. 1B. Dilation catheter 102 may then be moved proximal relative to hollow catheter 114 such that at least a portion of the proximal portion of expandable portion 108 is received into hollow catheter 114. While hollow catheter 114, dilator 103, and shunt 10 remain stationary relative to the atrial septum, sheath 110 may be retracted proximally to expose the distal portion of shunt 10 such that the distal portion of shunt 10 deploys within the left atrium. Shunt 10 may be maintained stationary relative to the atrial septum using devices within sheath 110 such as those described in WO2020202046, the entire contents of which is incorporated by reference herein. For example, a device having a plurality of hooks may be used to engage with the proximal portion of shunt 10 within sheath 110.

After the distal portion of shunt 10 is deployed within the left atrium, delivery device 100 may be retracted proximally until the distal portion of shunt 10 contacts the atrial septal wall. Then, sheath 110 may be further retracted proximally while shunt 10 is maintained stationary relative to the atrial septum until the proximal portion of shunt 10 is exposed from distal region 112 of sheath 110 and deploys within the right atrium of the patient as shown in FIG. 1C. Delivery device 100 may then be removed from the patient, leaving shunt 10 implanted at the atrial septum.

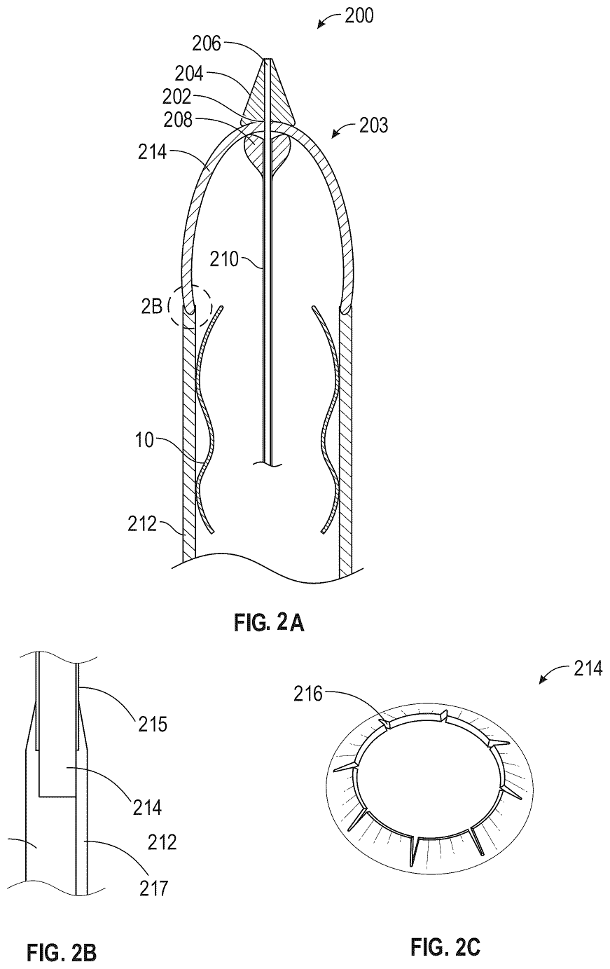

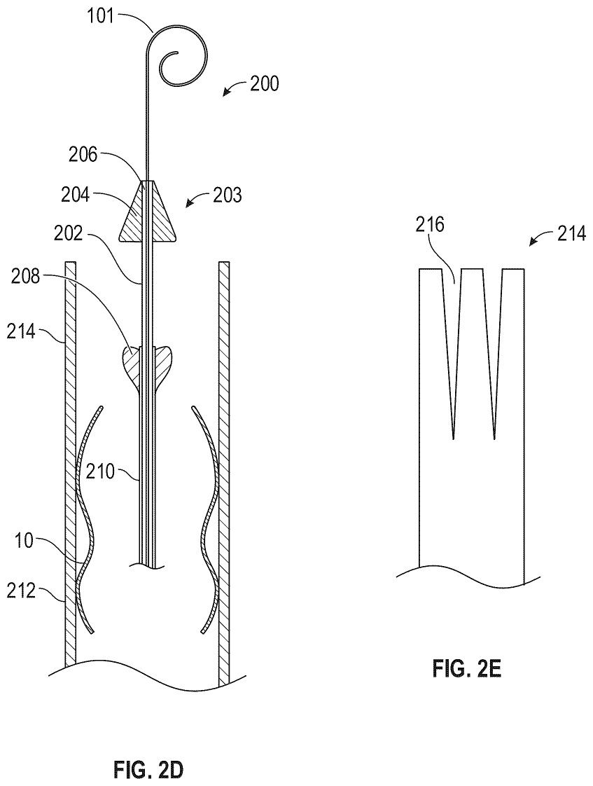

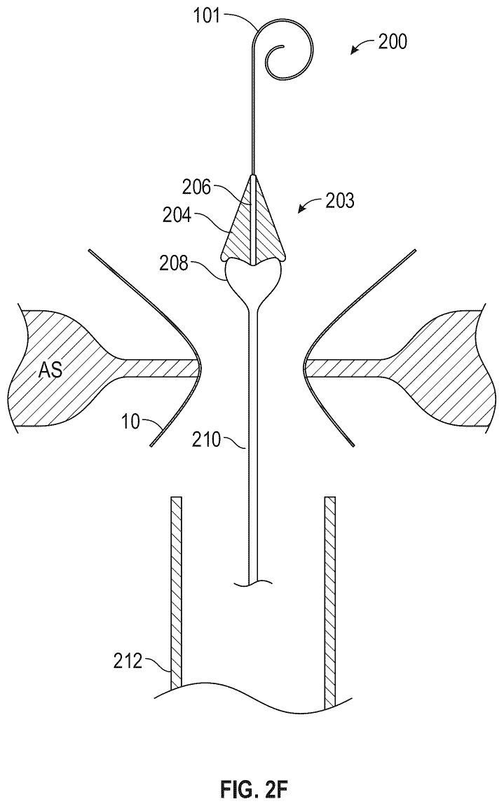

Referring now to FIGS. 2A to 2F, exemplary delivery device 200 for delivering interatrial shunt device 10 to the atrial septum is provided. As shown in FIGS. 2A and 2D, delivery device 200 includes sheath 212 removeably coupled to dilator 203. Dilator 203 includes proximal portion 208 coupled to outer tube 210, and distal portion 204 coupled to inner tube 202 moveably disposed within outer tube 210. Dilator 203 may be expandable, such that proximal portion 208 may be moved relative to distal portion 204, e.g., via actuation of a handle external to the patient that is independently coupled to outer tube 210 and inner tube 202. As shown in FIG. 2A, the proximal surface of distal portion 204 may have a geometry corresponding to the distal surface of proximal portion 208. For example, distal portion 204 may have an arrowhead shape.

Distal portion 204 may be a low durometer soft tip and may have an atraumatic cone shape that may be inserted through a puncture of the atrial septum to enlarge the puncture without damaging the surrounding tissue. Alternatively, distal portion 204 may have a sharp needle tip that may be used to create the puncture within the atrial septum, such that further advancement of distal portion 204 across the atrial septum enlarges the puncture. Dilator 203 may have a guidewire lumen 206 sized and shaped to receive a guidewire therethrough, such that device 200 may be advanced over a conventional guidewire across the atrial septum. Accordingly, guidewire lumen 206 may extend through distal portion 204 and inner tube 202.

Sheath 212 has a lumen extending from distal region 214 of sheath 212 to the proximal region of the sheath external to the patient. The lumen of sheath 212 is sized and shaped to receive shunt 10 in its collapsed delivery state. In addition, outer tube 210 may be moveably disposed within the lumen of sheath 212, such that dilator 203 may be moved relative to sheath 212, e.g., via actuation of the handle that is independently coupled to outer tube 210, inner tube 202, and sheath 212.

Distal region 214 of sheath 212 may be formed of an clastic material, e.g., superelastic Nitinol, and may be transitionable between a contracted state and an expanded state where sheath 212 has a generally tubular shape. For example, distal region 214 may be heat-set during manufacturing in the expanded state, such that distal region 214 is biased toward the expanded state, as shown in FIG. 2E. Accordingly, prior to insertion into the patient, distal region 214 may be contracted and positioned between distal portion 204 and proximal portion 208 of dilator 203, as shown in FIG. 2A. For example, distal portion 204 and proximal portion 208 may initially be decoupled, e.g., spaced apart from each other, thereby providing a gap therebetween, and upon contraction of distal region 214 of sheath 212, such that when the distal end of distal region 214 is contracted radially inward toward inner tube 202, distal portion 204 and proximal portion 208 may be moved toward each other to sandwich distal region 214 therebetween, thereby maintaining distal region 214 in its contracted state. Moreover, as shown in FIG. 2B, at least distal region 214 of sheath 212 may be encapsulated with biocompatible material 215, e.g., polyether block amide (PEBA), such as PEBAX® (made available by Arkema, Colombes, France). As shown in FIG. 2B, a proximal portion of distal region 214, e.g., within the distal portion of sheath 212, may remained unencapsulated. As further shown in FIG. 2B, the inner surface of sheath 212 may be lined with layer 217, e.g., polytetrafluoroethylene (PTFE). Accordingly, at least a portion of sheath 212 and layer 217 may sandwich the proximal portion of distal region 214. Alternatively, in some embodiments, sheath 212 and distal region 214 may be formed of a unitary construction.

As shown in FIG. 2A, in its contracted state, distal region 214 may have a dome shape, such that the distal end of distal region 214 has a smaller inner diameter than the inner diameter of the portion of sheath 212 proximal to distal region 214. The curvature of distal region 214 may be selected such that there is a step-free transition between distal region 214 and distal portion 204 when distal region 214 is sandwiched between distal portion 204 and proximal portion 208 of dilator 203, thereby forming a continuous dilator.

FIG. 2C illustrates an example distal region of the sheath. As will be understood by a person having ordinary skill in the art, the axial length of distal region 214 may be longer, as shown in FIG. 2A. As shown in FIG. 2C, in its contracted state, distal region 214 may have a plurality of tapered slots, e.g., longitudinal slits 216, disposed circumferentially along distal region 214. Each of the plurality of longitudinal slits 216 extend from the distal end of distal region 214 toward the proximal region of sheath 212, and have a length selected such that the fingers formed therebetween may be crimped to fit snugly around dilator 203 between distal portion 204 and proximal portion 208 in the contracted state.

As shown in FIG. 2D, distal portion 204 and proximal portion 208 may be decoupled by either moving distal portion 204 distally relative to proximal portion 208, or moving proximal portion 208 proximally relative to distal portion 204, or both, thereby releasing/disengaging distal region 214 of sheath 212. Accordingly, distal region 214 will return to its natural, expanded tubular configuration, as shown in FIG. 2E. As shown in FIG. 2E, distal region 214 of sheath 212 is expanded along plurality of longitudinal slits 216 such that each of the longitudinal slits forms a V-shape. The width of the distal end of each finger between adjacent longitudinal slits may depend on the number of slits of distal region 214. For example, distal region 214 may have 2, 4, 8, or more longitudinal slits 216, forming an equal number of fingers such that each finger has a trapezoidal shape. In one embodiment, the distal ends of the fingers may be rounded or smoothed to prevent them from damaging shunt 10 during its delivery, or injuring tissue during withdrawal of the sheath from the patient. Shunt 10 may be deployed by retracting sheath 212 proximally relative to dilator 203, as shown in FIG. 2F, to thereby implant shunt 10 at atrial septum AS.

Delivery of shunt 10 using delivery device 200 described above may proceed as follows. First, shunt 10 may be collapsed to its collapsed delivery state within sheath 212 in a similar manner as described above with regard to sheath 110. Next, inner tube 202 may be received through the distal end of outer tube 210, and outer tube 210 may be advanced over inner tube 202 until proximal portion 208 is adjacent to distal portion 204. Dilator 203 may then be advanced through the lumen of sheath 212 until distal portion 204 and proximal portion 208 are in proximity of distal region 214 of sheath 212. Distal portion 204 and proximal portion 208 may be spaced apart enough such that distal region 214 may be contracted to its contracted state so that the distal end of distal region 214 is positioned between distal portion 204 and proximal portion 208. Distal portion 204 and proximal portion 208 may be moved toward each other to sandwich the distal end of distal region 214 therebetween, and locked in place. Inner tube 202, outer tube 210, and sheath 212 may each be coupled at their proximal regions to a handle for use by a clinician, such that each component may be independently actuated via the handle.

Guidewire 101 may be advanced to the target location through a puncture of the atrial septum, e.g., within the left atrium of the patient. Device 200 may be advanced over guidewire 101 via guidewire lumen 206 until distal portion 204 comes into contact with the puncture of the atrial septum. Device 200 may be further advanced such that dilator 203 enlarges the puncture of the atrial septum as the tissue surrounding the puncture is smoothly guided over distal portion 204, followed by distal region 214 and sheath 212. Unlike other known delivery systems, a separate dilator is not required to enlarge the puncture of the atrial septum, and subsequently removed.

Under visualization methods such as fluoroscopy and/or ultrasound imaging, the target position of device 200 relative to the atrial septum may be verified, e.g., via a radiopaque marker on sheath 212. Next, distal portion 204 and proximal portion 208 may be moved apart from each other, to thereby release/disengage distal region 214 such that distal region 214 expands to its expanded, tubular shape. While dilator 203 and shunt 10 remain stationary relative to the atrial septum, sheath 212 may be retracted proximally to expose the distal portion of shunt 10 such that the distal portion of shunt 10 deploys within the left atrium. Shunt 10 may be maintained stationary relative to the atrial septum using devices within sheath 212 such as those described in WO2020202046, the entire contents of which is incorporated by reference herein. For example, a device having a plurality of hooks may be used to engage with the proximal portion of shunt 10 within sheath 212.

After the distal portion of shunt 10 is deployed within the left atrium, delivery device 200 may be retracted proximally until the distal portion of shunt 10 contacts the atrial septal wall. Then, sheath 212 may be further retracted proximally while shunt 10 is maintained stationary relative to the atrial septum until the proximal portion of shunt 10 is exposed from distal region 214 of sheath 212 and deploys within the right atrium of the patient as shown in FIG. 2F. Delivery device 200 may then be removed from the patient, leaving shunt 10 implanted at the atrial septum.

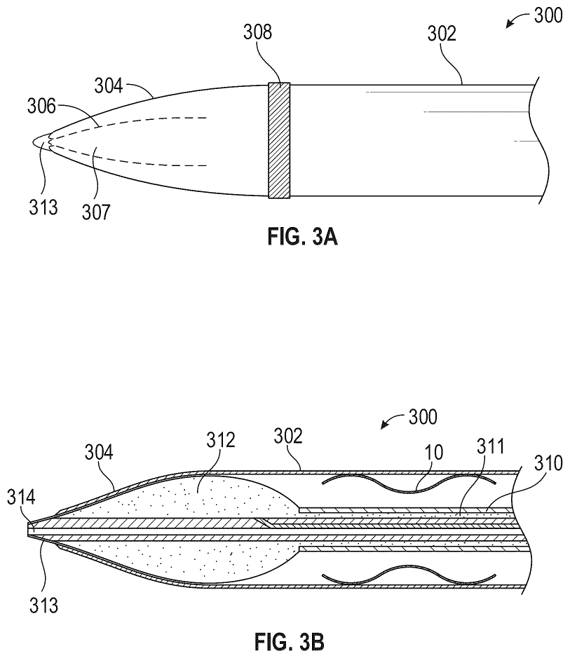

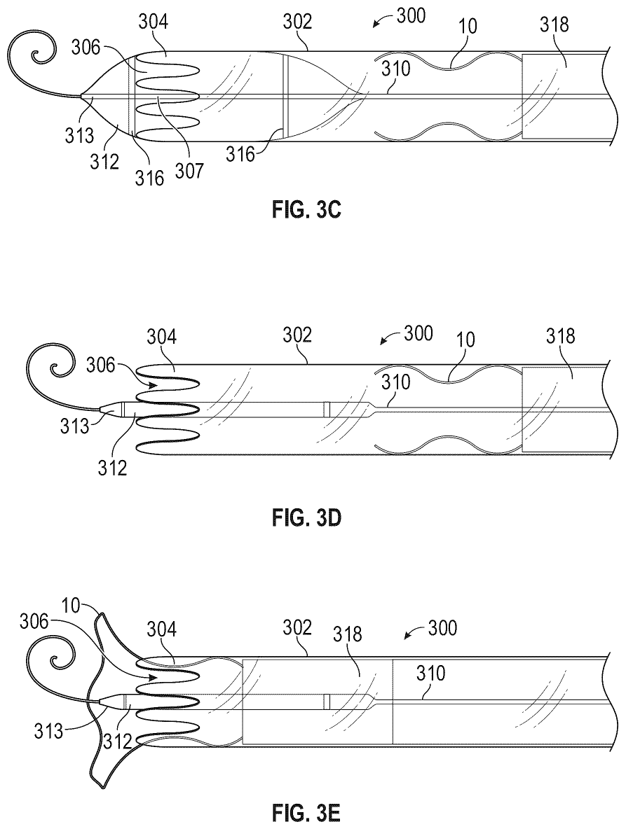

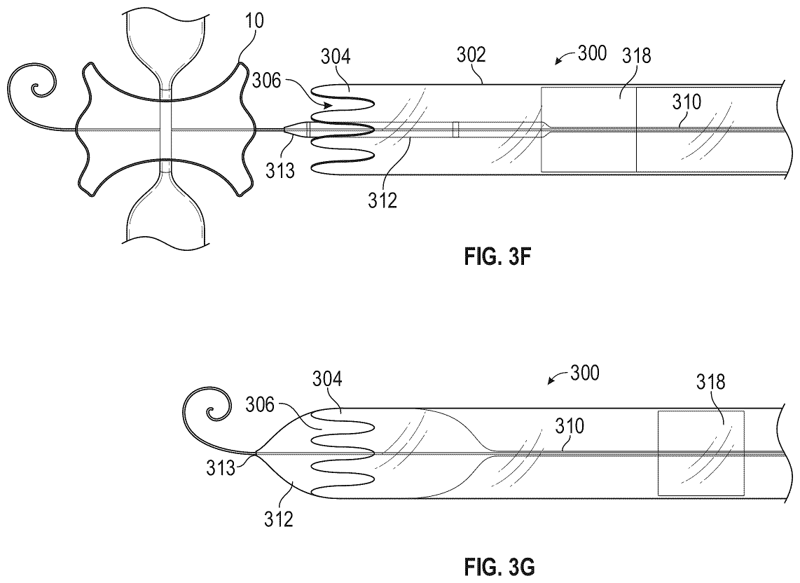

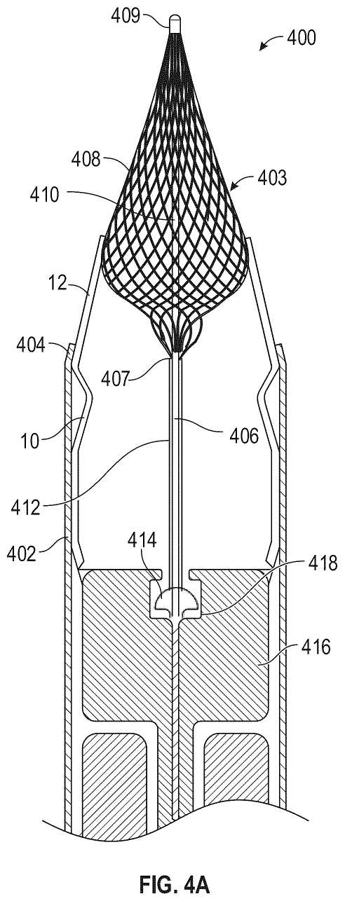

Referring now to FIGS. 3A to 3G, exemplary delivery device 300 for delivering interatrial shunt device 10 to the atrial septum is provided. Delivery device 300 includes sheath 302 and balloon catheter 310. Sheath 302 may have a lumen sized and shaped to receive balloon catheter 310 therein. As described in further detail below, balloon catheter 310 has inflatable balloon 312 disposed at its distal region. The lumen of sheath 302 is further sized and shaped to receive shunt 10 in its collapsed delivery state, and includes distal portion 304 having a plurality of longitudinal slits 306. Distal portion 304 may be formed of a malleable material, e.g., martensitic Nitinol or stainless steel, such that distal portion 304 is in its contracted state prior to delivery/deployment of shunt 10 at the atrial septum.

As shown in FIG. 3A, the distal end of fingers 307 formed by longitudinal slits 306 of sheath 302 may have a rounded shape and contact each other in the contracted state. The rounded shape of the distal end of fingers 307 define an opening when distal portion 304 is in its contracted state. Accordingly, as further shown in FIG. 3A, distal tip 313 of balloon 312 may be pointed, such that distal tip 313 protrudes through the opening formed by fingers 307, and thereby forming a continuous dilator with distal portion 304. The round shape of the distal end of fingers 307 of distal portion 304 assist in protecting balloon 312 and shunt 10 from damage during balloon expansion of fingers 307 as well as during deployment of shunt 10, as described in further detail below. In addition, as shown in FIG. 3A, sheath 302 further may include radiopaque marker 308 to assist in verification of delivery device 300 with respect to the atrial septum during delivery/deployment of shunt 10.

As shown in FIG. 3B, balloon catheter 310 has inflatable balloon 312 disposed at its distal region, and includes fluid lumen 311 for introducing fluid to balloon 312 to inflate/deflate balloon 312. Accordingly, as shown in FIG. 3B, balloon 312 may be positioned within the lumen of sheath 302 adjacent to longitudinal slits 306 in an inflated state, such that distal tip 313 of balloon 312 extends beyond the distal end of distal portion 304 when distal portion 304 is in its contracted state, thereby forming a continuous dilator. Balloon 312 may be inflated prior to delivery of delivery device 300 to the atrial septum, or alternatively, balloon 312 may be in a deflated state until delivery device 300 is delivered to the atrial septum, and then inflated. In the inflated state, balloon 312 may provide additional support to distal portion 304 during delivery of delivery device 300, as shown in FIG. 3B.

Moreover, delivery device 300 may include guidewire lumen 314 extending through balloon catheter 310, sized and shaped to receive a guidewire therethrough. Accordingly, device 300 may be advanced over guidewire 101 via guidewire lumen 314 until distal tip 313 of balloon 312 comes into contact with the puncture of the atrial septum. Device 300 may be further advanced such that distal tip 313 and distal portion 304 enlarges the puncture of the atrial septum as the tissue surrounding the puncture is smoothly guided over distal tip 313, followed by distal portion 304 and sheath 302.

Under visualization methods such as fluoroscopy and/or ultrasound imaging, the target position of device 300 relative to the atrial septum may be verified, e.g., via radiopaque marker 308 on sheath 302. Next, balloon catheter 310 may be advanced distally such that balloon 312, in its inflated state, pushes against distal portion 304, causing distal portion 304 to expand radially outward and transition to an expanded state, as shown in FIG. 3C. Radiopaque markers 316 on the outer surface of balloon 312 may be used to visualize balloon 312 under fluoroscopy relative to sheath 302 to ensure that distal portion 304 is sufficiently expanded. Balloon 312 may then be deflated, as shown in FIG. 3D, while distal portion 304 remains in its expanded state.

Shunt 10 may be maintained stationary relative to the atrial septum using devices within sheath 302 such as those described in WO2020202046, the entire contents of which is incorporated by reference herein. For example, pusher 318 may have a plurality of hooks that may be used to engage with the proximal portion of shunt 10 within sheath 302. Next, pusher 318 slidably disposed within the lumen of sheath 302, may be advanced distally relative to sheath 302 to push shunt 10 distally through the lumen of sheath 302 until the distal portion of shunt 10 is exposed beyond distal portion 304 such that the distal portion of shunt 10 deploys within the left atrium, as shown in FIG. 3E.

After the distal portion of shunt 10 is deployed within the left atrium, delivery device 300 may be retracted proximally until the distal portion of shunt 10 contacts the atrial septal wall. Then, sheath 302 may be further retracted proximally while shunt 10 is maintained stationary relative to the atrial septum until the proximal portion of shunt 10 is exposed from distal portion 304 of sheath 302 and deploys within the right atrium of the patient as shown in FIG. 3F. Next, balloon 312 may be re-advanced through sheath 302 and re-inflated such that balloon 312 is adjacent to distal portion 304 to thereby re-form a continuous device, as shown in FIG. 3G. Delivery device 300 may then be removed from the patient, leaving shunt 10 implanted at the atrial septum.

Alternatively, in some embodiments, distal portion 304 may be formed of a shape-memory material, e.g., martensitic Nitinol, with an austenitic finish (AF) temperature above body temperature. Thus, distal portion 304 may be heat set to its contracted state for delivery of delivery device 300. Balloon 312 may then be advanced distally relative to sheath 302 to expand distal portion 304 to its expanded state as described above, while distal portion 304 is in its martensitic phase. After shunt 10 is deployed at the atrial septum as described above, distal portion 304 may be transitioned back to its contracted state by exposing distal portion 304 to heat. For example, heated saline having a temperature above the AF temperature of distal portion 304 may be injected through sheath 302 to transmit heat to distal portion 304, to thereby cause distal portion 304 to transition from its expanded state to its contracted state. Delivery device 300 may then be removed from the patient, leaving shunt 10 implanted at the atrial septum.

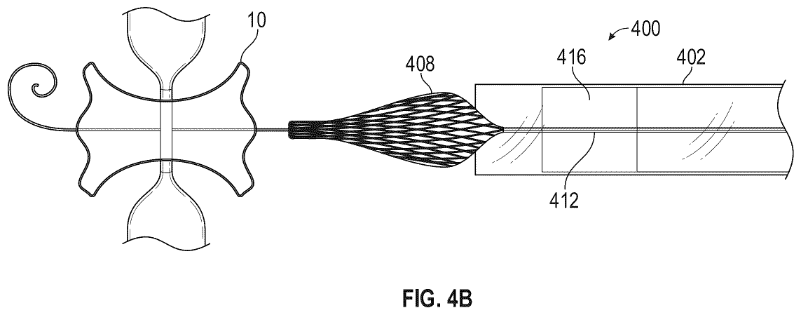

Referring now to FIGS. 4A and 4B, exemplary delivery device 400 for delivering interatrial shunt device 10 to the atrial septum is provided. As shown in FIG. 4A, delivery device 400 includes sheath 402 and dilator 403. Distal portion 12 of shunt device 10 may be used as part of delivery device 400 to facilitate dilation of the puncture of the atrial septum as described in further detail below. Sheath 402 has a lumen extending from the distal region of sheath 402 to the proximal region of the sheath external to the patient. The lumen of sheath 402 is sized and shaped to receive at least a portion of shunt 10 in its collapsed delivery state.

Dilator 403 includes expandable braided tip 408 that may be formed of a wire mesh, and may transition between an expanded state and a contracted state. For example, proximal portion 407 may be coupled to a distal end of outer tube 412, such that proximal portion 407 may be actuated via outer tube 412 and piston 416. As shown in FIG. 4A, piston 416 may include cavity 418 sized and shaped to engage with pin 414 at the proximal end of outer tube 412. Accordingly, actuation of piston 416, e.g., via actuation of a handle external to the patient that is independently coupled to piston 416, will push or pull outer tube 412 via pin 414. Moreover, distal portion 409 may be coupled to inner tube 410 moveably disposed within outer tube 412 and pin 414, such that proximal portion 407 may be moved relative to distal portion 409, e.g., via actuation of a handle external to the patient that is independently coupled to piston 416 and inner tube 410, to thereby transition braided tip 408 between its expanded and contracted state. For example, braided tip 408 will expand as distal portion 409 and proximal 407 are moved closer together, and will contract as distal portion 409 and proximal 407 are moved farther apart. Outer tube 412 and inner tube 410 may be concentric tubes, e.g., PEEK tubes.

In addition, piston 416 may engage with and maintain shunt 10 in its collapsed delivery state, and may maintain shunt 10 stationary relative to the atrial septum. For example, piston 416 may include a plurality of hooks that may be used to engage with the proximal portion of shunt 10 within sheath 402.

As shown in FIG. 4A, in the expanded state, the distal portion of braided tip 408 may form an atraumatic cone shape that may be inserted through a puncture of the atrial septum to enlarge the puncture without damaging the surrounding tissue. Accordingly, the wire mesh of braided tip 408 may be encapsulated with a biocompatible material to facilitate with the dilation of the puncture of the atrial septum. Alternatively, the distal portion of braided tip 408 may have a sharp needle tip that may be used to create the puncture within the atrial septum, such that further advancement of braided tip 408 across the atrial septum enlarges the puncture. Dilator 403 may have a guidewire lumen 406 extending through inner tube 410, sized and shaped to receive a guidewire therethrough, such that device 400 may be advanced over a conventional guidewire across the atrial septum. Accordingly, guidewire lumen 406 may extend through braided tip 408 and inner tube 410.

As shown in FIG. 4A, distal portion 12 of shunt device 10 may be used as part of delivery device 400 to facilitate dilation of the puncture of the atrial septum. For example, distal portion 12 of shunt device 10 may be formed of an shape-memory material, e.g., martensitic Nitinol with an austenitic finish temperature, Af, greater than body temperature, e.g. greater than 45 degrees Celsius, and may be heat-set in an expanded configuration. Further, distal portion 12 may be crimped into a collapsed dilator state, as shown in FIG. 4A. In its collapsed dilator state, distal portion 12 of shunt device 10 may contact the outer surface of braided tip 408, preferably at a point along the outer surface of braided tip 408 where the cross-sectional area of braided tip increases from distal portion 409 toward proximal portion 407. Accordingly, braided tip 408 may be expanded such that there is a step-free transition between braided tip 408 and distal portion 12 of shunt device 10, thereby forming a continuous dilator. Moreover, as shown in FIG. 4A, distal region 404 of sheath 402 may have a geometry that facilitates a step-free transition between sheath 402 and distal portion 12 of shunt device 10. For example, distal region 404 of sheath 402 may be curved radially inward to engage with distal portion 12 of shunt device 10.

Distal portion 12 of shunt device 10 may be transitioned from its collapsed dilator state to an expanded deployed state, e.g., via the application of heat. For example, a warm fluid such a saline may be introduced over distal portion 12 of shunt device 10 to thereby heat distal portion 12 above a predetermined Af transition temperature, and cause distal portion 12 to expand to its heat-set expanded deployed state. The warm fluid may be introduced within sheath 402, exterior to outer tube 412. Alternatively or additionally, sheath 402 further may include one or more fluid channels 419 extending through piston 416 and coupled to a source of fluid external to the patient for introducing warm fluid across distal portion 12.

Delivery of shunt 10 using delivery device 400 described above may proceed as follows. First, distal portion 12 of shunt device 10 may be crimped to a collapsed dilator state, and the remainder of shunt 10 may be crimped to its collapsed delivery state within sheath 402, as shown in FIG. 4A, in a similar manner as described above with regard to sheaths 110, 212. The axially position of shunt 10 within sheath 402 may be adjusted such that distal portion 12 of shunt 10 is exposed from the distal end of distal region 404 of sheath 402.

Next, dilator 403 may then be advanced through the lumen of sheath 402 until braided tip 408 is adjacent to distal portion 12 of shunt device 10. Braided tip 408 may be expanded to its expanded state via actuation of inner tube 410 and piston 416, and accordingly, outer tube 412, as described above, to form a step-free transition between distal portion 12 of shunt device 10 and braided tip 408. For example, inner tube 410, piston 416, and sheath 402 may each be coupled at their proximal regions to a handle for use by a clinician, such that each component may be independently actuated via the handle.

Guidewire 101 may be advanced to the target location through a puncture of the atrial septum, e.g., within the left atrium of the patient. Device 400 may be advanced over guidewire 101 via guidewire lumen 406 until distal portion 409 of braided tip 408 comes into contact with the puncture of the atrial septum. Device 400 may be further advanced such that dilator 403 enlarges the puncture of the atrial septum as the tissue surrounding the puncture is smoothly guided over braided tip 408, followed by distal portion 12 of shunt device 10 and sheath 402. Unlike other known delivery systems, a separate dilator is not required to enlarge the puncture of the atrial septum, and subsequently removed.

Under visualization methods such as fluoroscopy and/or ultrasound imaging, the target position of device 400 relative to the atrial septum may be verified, e.g., via a radiopaque marker on sheath 402. Braided tip 408 may be contracted via movement of inner tube 410 relative to outer tube 412 as described above. Next, a warm fluid may be introduced across distal portion 12 of shunt device 10 to transition distal portion 12 from its collapsed dilator state to its expanded deployed state within the left atrium. Shunt 10 may be maintained stationary relative to the atrial septum via piston 416.

After the distal portion of shunt 10 is deployed within the left atrium, delivery device 400 may be retracted proximally until the distal portion of shunt 10 contacts the atrial septal wall. Then, sheath 402 may be further retracted proximally while shunt 10 is maintained stationary relative to the atrial septum until the proximal portion of shunt 10 is exposed from distal region 404 of sheath 402 and deploys within the right atrium of the patient. Delivery device 400 may then be removed from the patient, leaving shunt 10 implanted at the atrial septum.

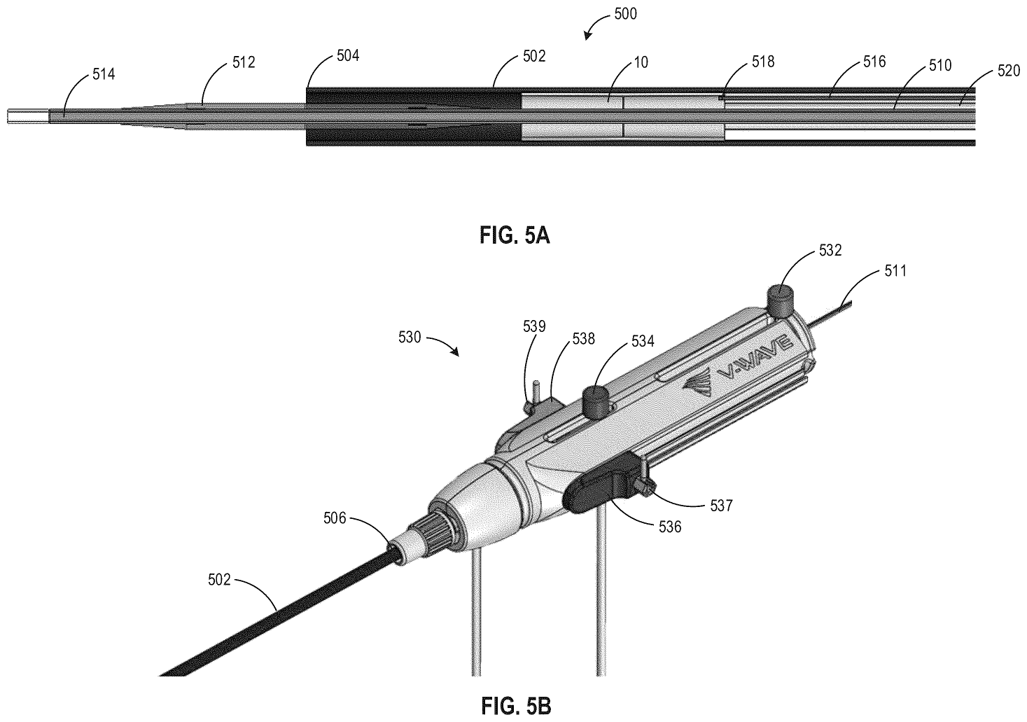

Referring now to FIGS. 5A and 5B, exemplary delivery device 500 operatively coupled to handle 530 for delivering interatrial shunt device 10 to the atrial septum is provided. As shown in FIG. 5A, delivery device 500 includes sheath 502, a dilator, e.g., balloon catheter 510, slidably disposed within the lumen of sheath 502, release knot 516 for releasably coupling to shunt 10 at knot connection 518 within the lumen of sheath 502, and pusher 520 slidably disposed within the lumen of sheath 502. For example, release knot 516 may be a Dyneema wire/cord. The lumen of sheath 502 may be sized and shaped to receive shunt 10 in its collapsed delivery state. Distal region 504 of sheath 502 may be linear or may have geometry that facilitates a step-free transition between distal region 504 and balloon 512 to form a smooth and continuous dilator when balloon 512 is in its expanded state, as described in further detail below. For example, distal region 504 of sheath 502 may be curved radially inward to engage with the outer surface of balloon 512. Moreover, delivery device 500 may include guidewire lumen 514 extending through balloon catheter 510, sized and shaped to receive a guidewire therethrough.

Each of sheath 502, balloon catheter 510, release knot 516, and pusher 520 may be operatively coupled to handle 530, such that they are all independently actuatable relative to each other. For example, as shown in FIG. 5B, proximal region 506 of sheath 502 may be coupled to handle 530, balloon catheter 510 may be operatively coupled to actuator 532 of handle 530 which may be actuated to move balloon catheter 510 axially relative to sheath 502, pusher 520 may be operatively coupled to actuator 534 of handle 530 which may be actuated to move pusher 520 axially relative to sheath 502, a first end of release knot 516 may be operatively coupled to release actuator 536 of handle 530 which may be actuated to disassemble knot connection 518 and disengage release knot 516 from shunt 10, and a second end of release knot 516 may be operatively coupled to retrieval actuator 538 of handle 530 which may be actuated to retract release knot 516, and accordingly shunt 10 via knot connection 518, within the lumen of sheath 502. In some embodiments, to prevent accidental disengagement between release knot 516 and shunt 10, release actuator 536 may include lock 537 which may be actuated to transition between a locked configuration where release actuator 536 may not be actuated relative to handle 530, and an unlocked configuration where release actuator 536 may be actuated and moved along handle 530. Moreover, to prevent accidently half-way retrieval of shunt 10, retrieval actuator 538 may include lock 539 which may be actuated to transition between a locked configuration where retrieval actuator 538 may not be actuated relative to handle 530, and an unlocked configuration where retrieval actuator 538 may be actuated and moved along handle 530.

Referring again to FIG. 5A, balloon catheter 510 may include inflatable balloon 512 disposed at its distal region. Balloon 512 is configured to transition between a deflated, compressed state and an inflated, expanded state. Accordingly, balloon catheter 510 may include a fluid lumen fluidically coupled to a fluid source for introducing fluid to balloon 512 to inflate/deflate balloon 512. In addition, balloon 512 may be formed having a tapered cone shape at its distal end. Balloon 512 may have a symmetric profile, such that both its proximal and distal ends have a tapered cone shape when balloon 512 is in its expanded state. When balloon 512 is positioned adjacent to distal region 504 of sheath 502, in its expanded state, the outer surface of balloon 512 may form a step-free transition between distal region 504 and balloon 512 to form a smooth and continuous dilator.

Moreover, pusher 520 may be a multi-lumen catheter slidably disposed within the lumen of sheath 502, having a distal end configured to engage with shunt 10 in its collapsed delivery state within the lumen of sheath 502, e.g., via actuation of actuator 534. For example, pusher 520 may have a first lumen sized and shaped to slidably receive balloon catheter 510 (including balloon 512 in its collapsed state) therethrough, and one or more lumens sized and shaped to slidably receive one or both ends of release knot 516 therethrough. For example, pusher 520 may have a single lumen sized and shaped to slidably receive both ends of release knot 516 therethrough, or alternatively, pusher 520 may have one lumen sized and shaped to slidably receive a first end of release knot 516 operatively coupled to release actuator 536 therethrough, and another lumen sized and shaped to slidably receive a second end of release knot 516 operatively coupled to retrieval actuator 538 therethrough. The lumen of pusher 520 that slidably receives balloon catheter 510 may be coaxial with the longitudinal axis of sheath 502, and the one or more lumens that slidably receive the ends of release knot 516 may not be coaxial with the longitudinal axis of sheath 502. Accordingly, pusher 520 may be advanced distally relative to sheath 502 and balloon catheter 510 via actuation of actuator 534 to push shunt 10 distally through the lumen of sheath 502 until at least the distal portion of shunt 10 is exposed beyond distal region 504 and transitions to its expanded deployed state, e.g., within the left atrium.

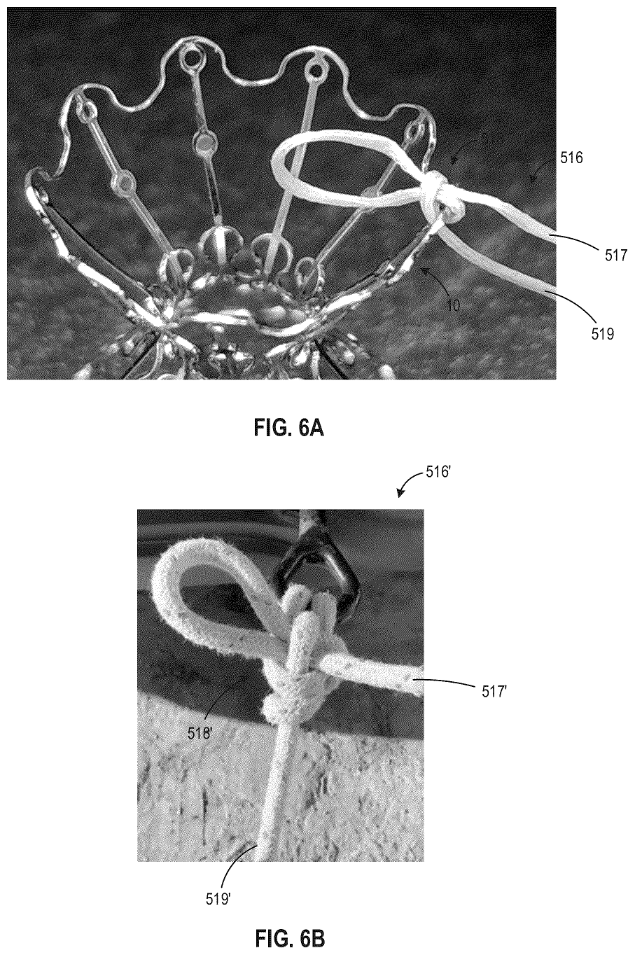

As described above, release knot 516 may be releasably coupled to shunt 10 in its collapsed delivery state within the lumen of sheath 502 via knot connection 518. FIG. 6A illustrates an exemplary knot mechanism for releasably coupling release knot 516 to shunt 10. As shown in FIG. 6A, release knot 516 may be formed of a single force transmission element, e.g., a Dyneema wire/cord, having a release wire portion, e.g., release end 517, knot connection 518, and a standing portion, e.g., retrieval end 519. For example, release knot 516 may be tied to shunt 10, e.g., at the proximal portion of shunt 10, to form knot connection 518, such that release end 517 and retrieval end 519 extends therefrom. Knot connection 518 may be a knot such as a painters hitch or Quick Tie and Release (QTaR) hitch, such that application of a retraction force to release end 517, e.g., via release actuator 536, causes release end 517 to pull knot connection 518 in a manner that causes knot connection 518 to disassemble and disengage release knot 516 from shunt 10, whereas application of a retraction force to retrieval end 519, e.g., via retrieval actuator 538, causes retrieval end 519 to pull knot connection 518 to thereby pull shunt 10 within the lumen of sheath 502. Accordingly, release knot 516 may have a predetermined amount of excess length, e.g., slack, disposed within device 800, e.g., within sheath 502/pusher 520 distal to handle 520, such that when shunt 10 is moved distally within sheath 502 via pusher 520, release knot 516, which is coupled to shunt 10 via knot connection 518, also may move distally within sheath 502 without applying force to release actuator 536 or retrieval actuator 538. Thus, actuation of release actuator 536 and/or retrieval actuator 538 may not disengage or halfway retrieve shunt 10 until shunt 10 is halfway deployed from sheath 502, e.g., when the slack of release knot 516 is removed.

As will be understood by a person having ordinary skill in the art, the knot configuration illustrated in FIG. 6A is one example of numerous knot configurations suitable for use with the delivery devices described herein, in accordance with the principles of the present disclosure. For example, FIG. 6B illustrates another exemplary release knot 516′ having a release wire portion, e.g., release end 517′, knot connection 518′, and a standing portion, e.g., retrieval end 519′. Knot connection 518′ may have a configuration similar to that of knot connection 518, except that knot connection 518′ includes an additional loop to provide additional securement between release knot 516′ and the shunt.

Moreover, the release knots described herein may be used to facilitate transitioning of shunt 10 from its expanded deployed state toward its collapsed delivery state, for example, as described in U.S. Pat. No. 10,940,296 to Keren, assigned to the assignee of the present invention, the entire contents of which are incorporated herein by reference. For example, in some embodiments, the release knot may be coupled to a proximal portion of shunt 10 and both the retrieval and release ends may be woven through two or more loops of the proximal end of shunt 10, e.g., at points evenly spaced around the circumference of the proximal end of shunt 10, such that retraction of the retrieval end of the release knot, e.g., through the lumen of pusher 520, applies an inward force to the proximal portion of shunt 10 to thereby collapse the proximal portion of shunt 10 radially inward to its collapsed delivery state; whereas, retraction of the release end of the release knot causes the knot connection to disassemble and disengages the release knot from shunt 10. Accordingly, the release knot may remain coupled to shunt 10 when shunt 10 is fully deployed at the atrial septum, e.g., by having enough slack within delivery device 500 distal to handle 530. Once shunt 10 is satisfactorily deployed, the release knot may be disengaged from shunt 10 by retraction of the release end of the release knot, and the entire release knot, including both the release and retrieval ends, may be pulled back into delivery sheath 502. If the deployment of shunt 10 is unsatisfactory, the retrieval end of the release knot may be retracted proximally to transition the proximal portion of shunt 10 toward its collapsed delivery state, and shunt 10 may be further retracted back into the lumen of sheath 502 via retraction of the retrieval end of the release knot for redeployment or removal.

Additionally or alternatively, the release knot may be coupled to a middle portion of shunt 10, e.g., the neck region between the proximal and distal flared end regions of shunt 10, and both the retrieval and release ends may be looped around the outer surface of the middle portion of shunt 10 toward the knot connection. Once shunt 10 is satisfactorily deployed, the release knot may be disengaged from shunt 10 by retraction of the release end of the release knot, and the entire release knot, including both the release and retrieval ends, may be pulled back into delivery sheath 502. If the deployment of shunt 10 is unsatisfactory, shunt 10 may be retrieved back into delivery sheath 502 by retraction of the retrieval end of the release knot proximally, e.g., through the lumen of pusher 520, which applies an inward force to the middle portion of shunt 10 to thereby collapse the middle portion of shunt 10 radially inward toward its collapsed delivery state. Collapsing the middle portion of shunt 10 also may cause the proximal portion of shunt 10 to at least partially collapse, such that shunt 10 may then be further retracted back into the lumen of sheath 502 via retraction of the retrieval end of the release knot for redeployment or removal.

Alternatively, the release knot may be coupled to a proximal portion of shunt 10 and both the retrieval and release ends may be passed through an initial loop at the proximal end of shunt 10, then through the central passageway of shunt 10, and out of the central passageway and looped around the outer surface of the middle portion of shunt 10, and back towards the knot connection, such that retraction of the retrieval end of the release knot, e.g., through the lumen of pusher 520, applies an inward force to the middle portion of shunt 10 to thereby collapse the middle portion of shunt 10 radially inward toward its collapsed delivery state; whereas, retraction of the release end of the release knot causes the knot connection to disassemble and disengages the release knot from shunt 10. As will be understood by a person having ordinary skill in the art, more than one release knot may be coupled to the shunt, e.g., at locations evenly spaced around the circumference of the shunt, to facilitate transitioning of shunt 10 toward its collapsed delivery state for full and/or halfway retrieval.

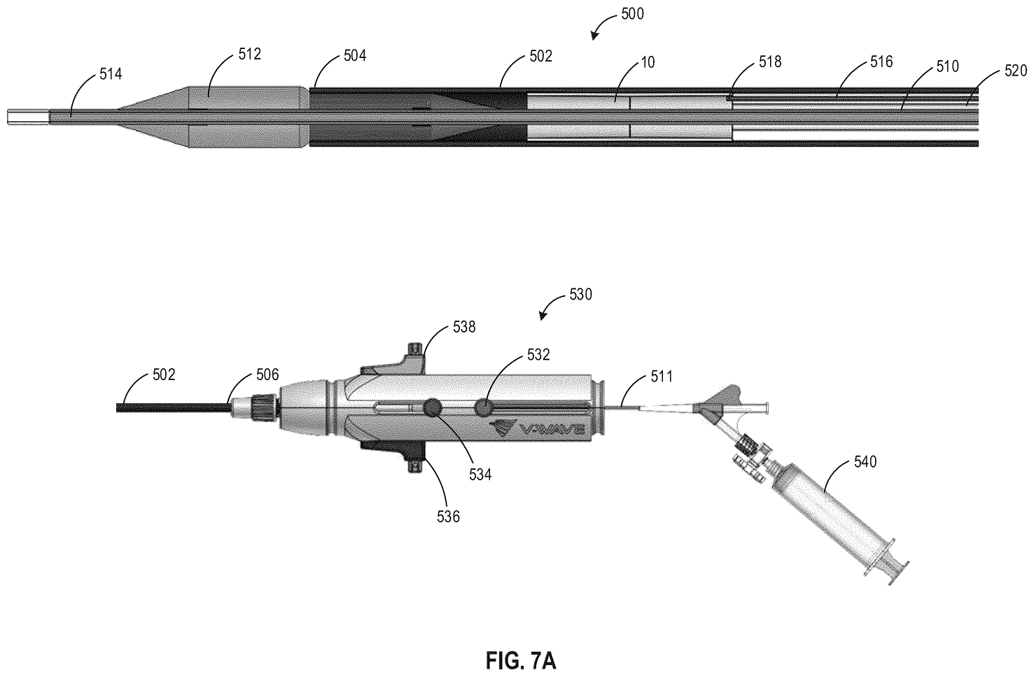

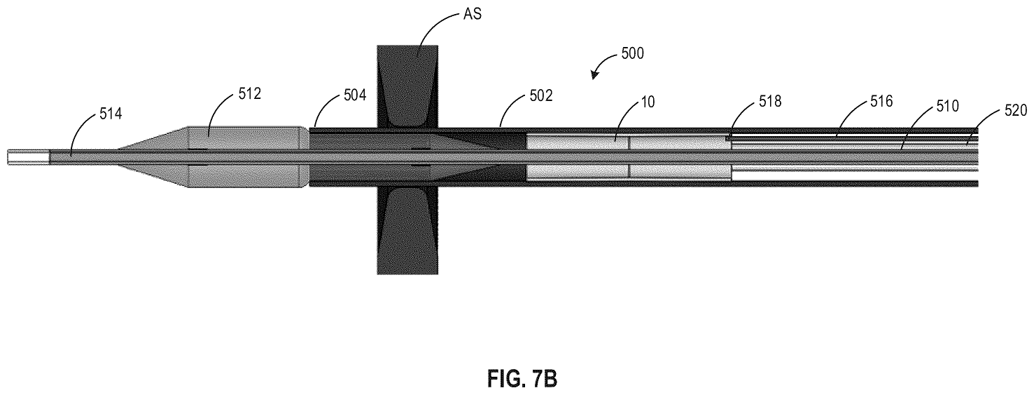

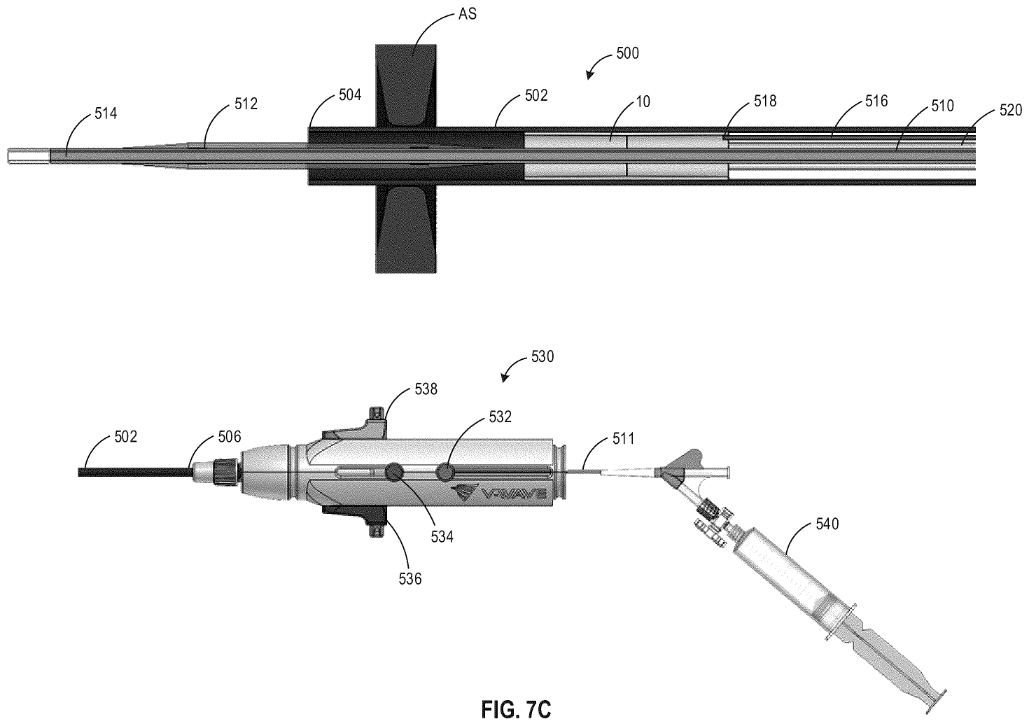

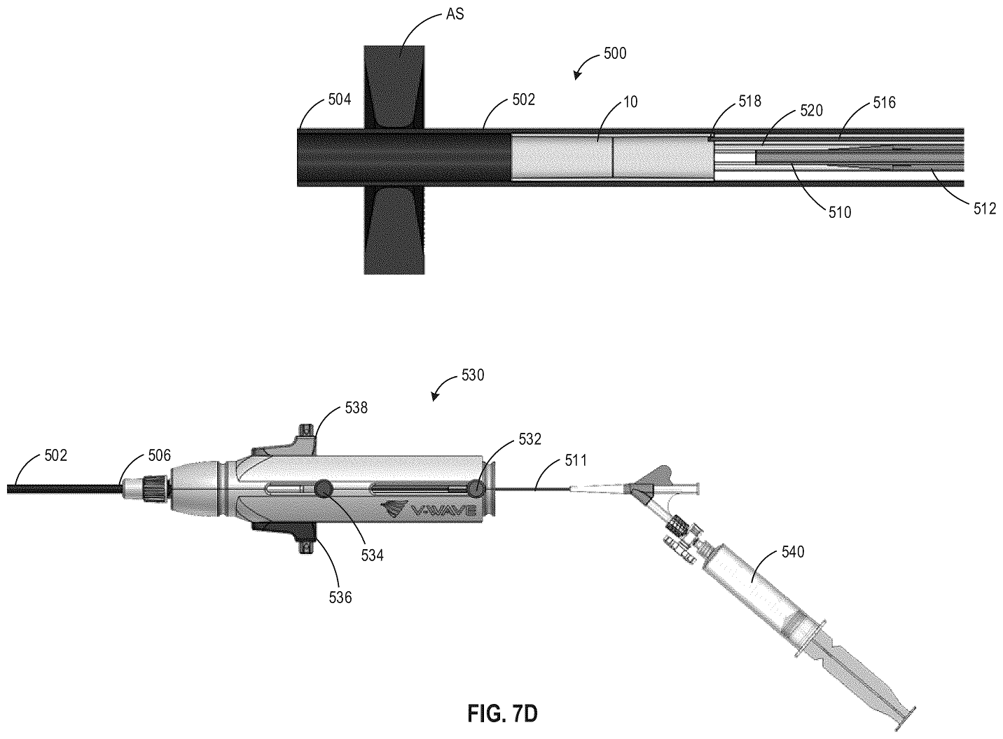

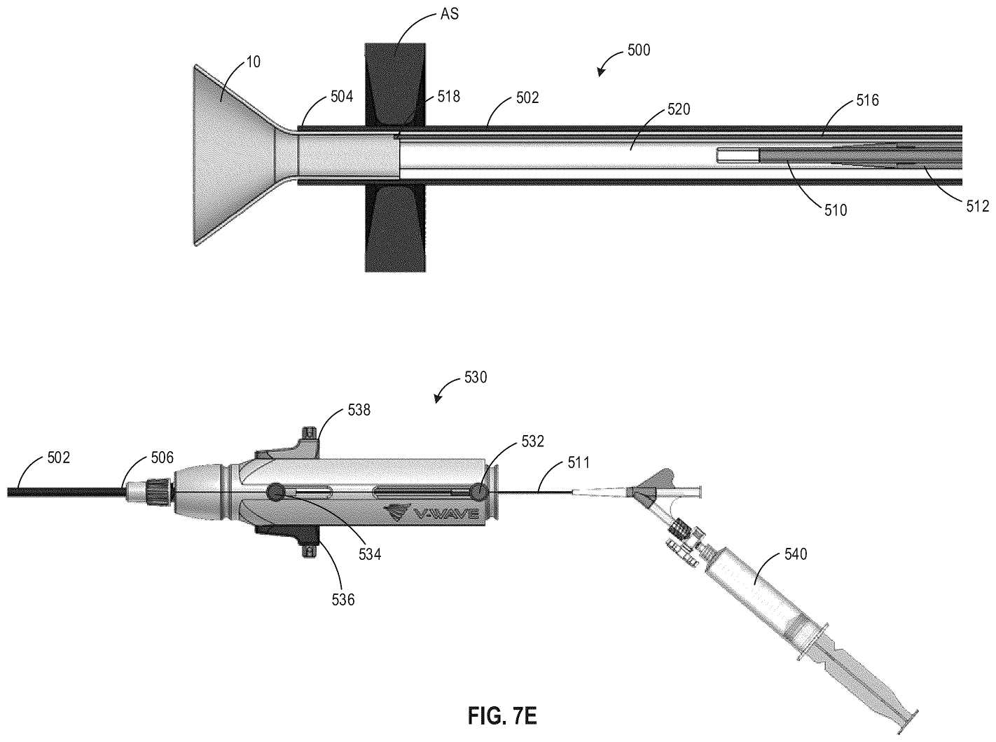

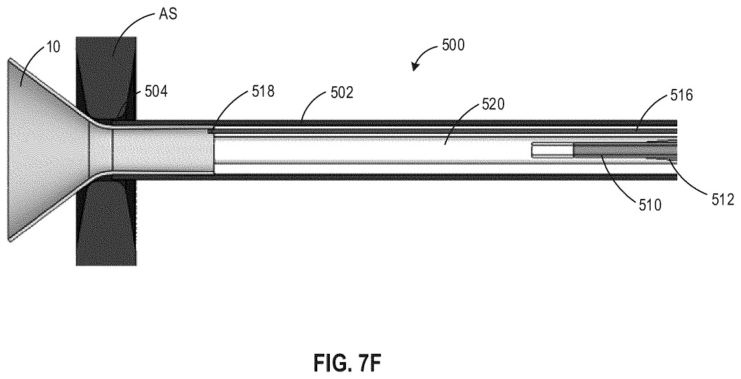

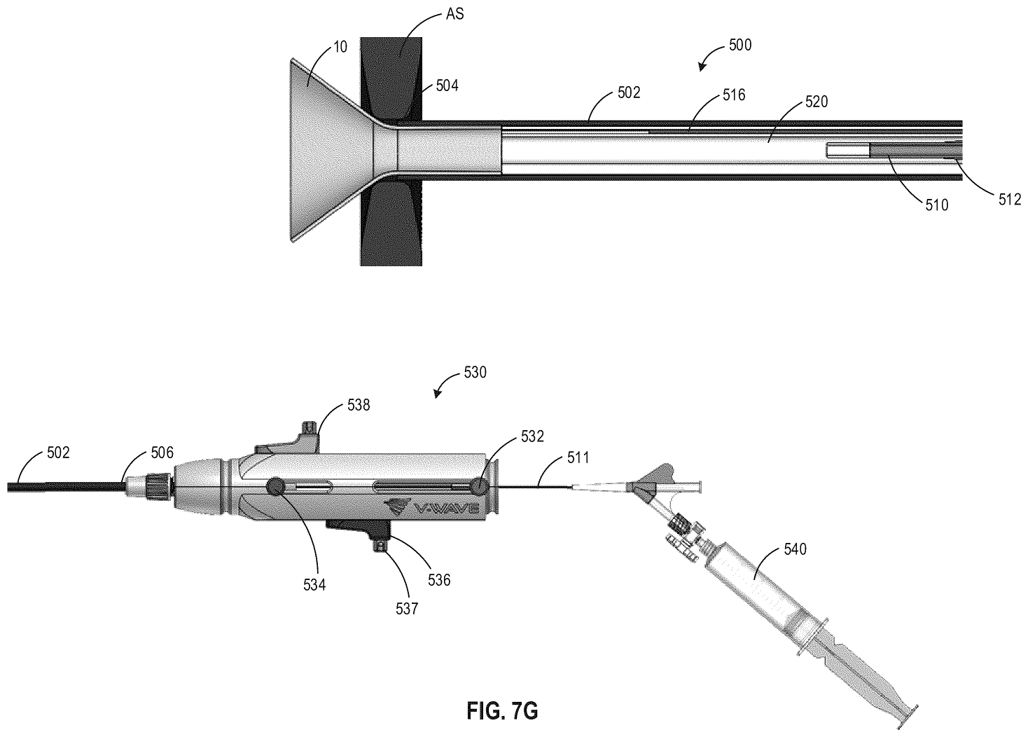

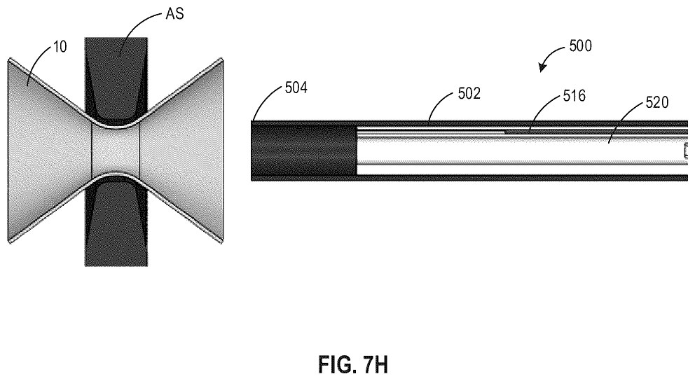

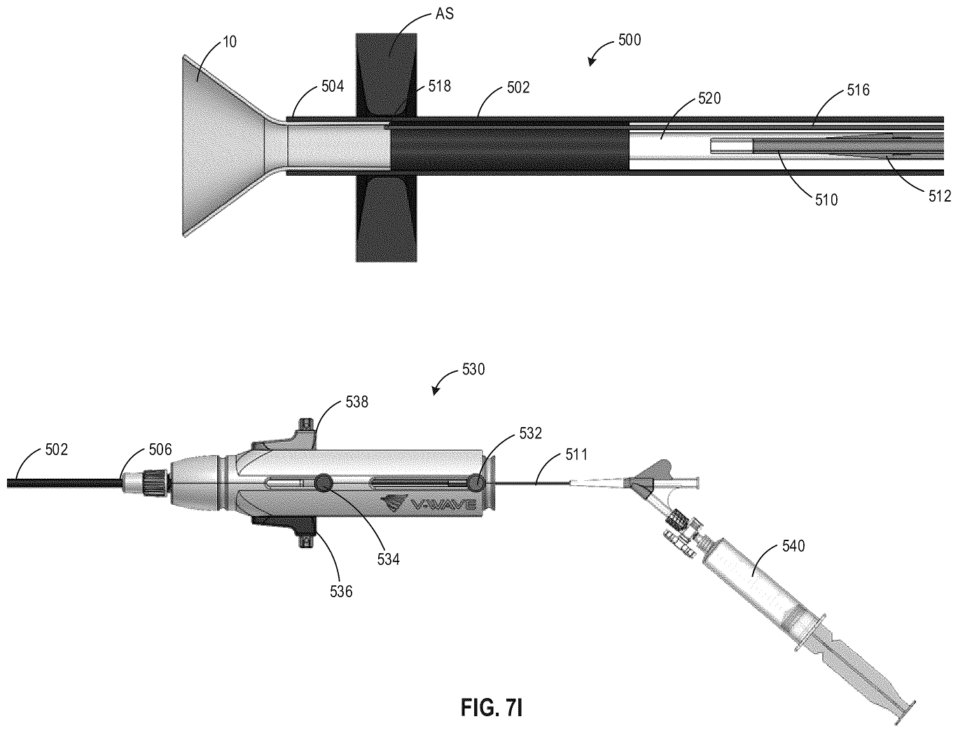

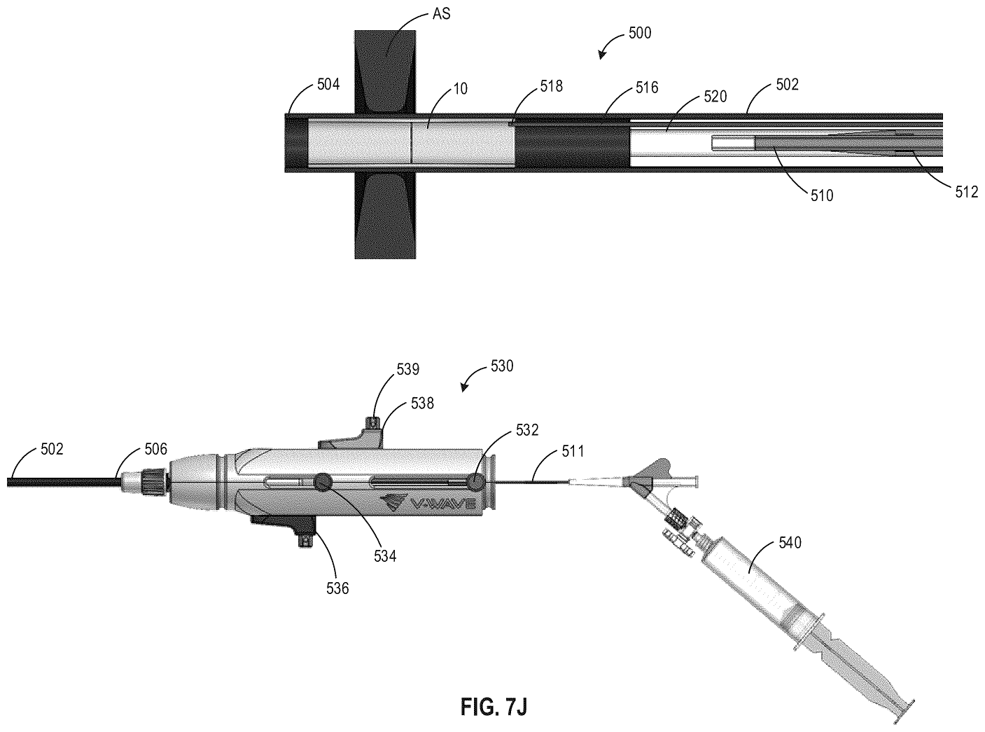

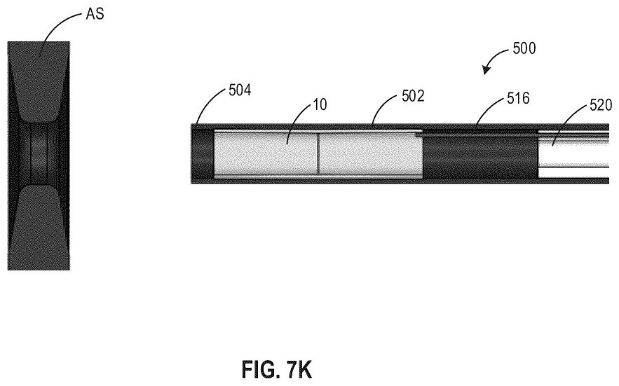

Referring now to FIGS. 7A to 7H, exemplary method steps for delivering shunt 10 to an implantation site within the atrial septum via delivery device 500 are provided. As shown in FIG. 7A, fluid source 540, e.g., a syringe pump, fluidically coupled to balloon 512 via fluid lumen 511 of balloon catheter 510 may be actuated to move a fluid into balloon 512 to thereby inflate balloon 512 from its deflated collapsed state to its inflated expanded state, such that in its expanded state, an outer surface of balloon 512 engages with the inner surface of the lumen of sheath 502 at distal region 504 of sheath 502, thereby forming a smooth and continuous dilator for delivering delivery device 500 to the atrial septum.

Device 500 with balloon 512 in its expanded state at distal region 504 of sheath 502 may be advanced over guidewire 101 via guidewire lumen 514 until the distal portion of balloon 512 comes into contact with the puncture of atrial septum AS. Device 500 may be further advanced such that balloon 512 enlarges/dilates the puncture of atrial septum AS as the tissue surrounding the puncture is smoothly guided over the distal portion of balloon 512, followed by distal region 504 of sheath 502, as shown in FIG. 7B. Under visualization methods such as fluoroscopy and/or ultrasound imaging such as trans-esophageal echo (TEE) or intracardiac echo (ICE), the target position of device 500 relative to atrial septum AS may be verified, e.g., via a radiopaque marker on sheath 502. Preferably, device 500 is positioned relative to atrial septum AS such that distal region 504 is spaced at least a predetermined distance from atrial septum AS within the left atrium to ensure full deployment of the distal portion of shunt 10 within the left atrium, as described in further detail below.

Next, as shown in FIG. 7C, balloon 512 may be deflated via fluid source 540, e.g., by actuating the syringe pump to withdraw fluid from balloon 512, such that balloon 512 transitions to its deflated, collapsed state. In some embodiments, shunt 10 may be maintained stationary relative to the atrial septum using devices within sheath 502 such as those described in WO2020202046. As shown in FIG. 7D, actuator 532 may then be actuated, e.g., moved from a first position on handle 530 proximally along handle 530 to a second position on handle 530, to thereby retract balloon catheter 510 and balloon 512 in its deflated state proximally relative to sheath 502. For example, balloon catheter 510 may be retracted proximally through shunt 10 in its collapsed, delivery state within sheath 502, until balloon 512 is disposed within the lumen of pusher 520, as shown in FIG. 7D. Accordingly, the lumen of sheath 502 may be unobstructed between shunt 10 and distal region 504.

Next, actuator 534 may be actuated, e.g., moved from a first position on handle 530 distally along handle 530 to a second position on handle 530, to thereby move pusher 520, and accordingly shunt 10, distally relative to sheath 502 until the distal portion of shunt 10 is exposed beyond distal region 504 of sheath 502 and deploys within the left atrium, as shown in FIG. 7E. For example, as pusher 520 is advanced distally within the lumen of sheath 502, the distal end of pusher 520 engages with the proximal end of shunt 10 in its collapsed delivery state, and pushes shunt 10 distally through the lumen of sheath 502. As described above, distal region 504 may be spaced from atrial septum AS at least a predetermined distance such that the distal portion of shunt 10 may fully deploy within the left atrium. Accordingly, device 500 may then be retracted relative to atrial septum AS, e.g., by moving handle 530 proximally, until the deployed distal portion of shunt 10 contacts atrial septum AS, as shown in FIG. 7F. The desired position of shunt 10 relative to atrial septum AS may be observed by the physician, e.g., via force feedback applied to device 500 via atrial septum AS, and/or may be visually verified via, e.g., fluoroscopy and/or ultrasound imaging such as trans-esophageal echo (TEE) or intracardiac echo (ICE).

As shown in FIG. 7G, release actuator 536 may be actuated, e.g., moved from a first position on handle 530 proximally along handle 530 to a second position on handle 530, to thereby retract release end 517 of release knot 516, which causes knot connection 518 to disassemble and disengage release knot 516 from shunt 10 within sheath 502. As described above, in some embodiments, lock 537 of release actuator 536 may be required to transition from its locked configuration to its unlocked configuration prior to moving release actuator 536 from the first position to the second position. Next, when shunt 10 is disengaged from release knot 516, device 500 may be further retracted proximally relative to atrial septum AS, e.g., by moving handle 530 proximally, such atrial septum AS applies a force to the deployed distal portion of shunt 10 to maintain shunt 10 in position relative to atrial septum AS as device 500 is retracted proximally until the proximal portion of shunt 10 is exposed beyond distal region 504 of sheath 502 and deploys within the right atrium, thus completing full deployment of the shunt, as shown in FIG. 7H.

After shunt 10 is fully deployed at atrial septum AS, device 500 may be removed from the patient, leaving shunt 10 implanted at the atrial septum. In some embodiments, prior to removal, actuator 532 may be actuated to move balloon catheter 510 distally within sheath 502, such that balloon 512 is positioned within sheath 502 adjacent to distal region 504, and balloon 512 may be inflated to its expanded state, e.g., via fluid source 540, to form a continuous dilator with distal region 504, as described above.

Referring now to FIGS. 7I to 7K, exemplary method steps for halfway retrieval of shunt 10 during delivery of shunt 10 at atrial septum AS are provided. For example, after the distal portion of shunt 10 is deployed, e.g., within the left atrium as shown in FIG. 7E or in another improper location, it may be desirable to transition shunt 10 back to its collapsed delivery state within sheath 502 and retrieve shunt 10. Accordingly, as shown in FIG. 7I, actuator 534 may be actuated, e.g., moved from the second position on handle 530 proximally along handle 530 to the first position on handle 530, to thereby move pusher 520 proximally relative to sheath 502, and provide an unobstructed pathway within the lumen of sheath 502 for shunt 10 to be disposed therein in its collapsed delivery state. Next, retrieval actuator 538 may be actuated, e.g., moved from a first position on handle 530 proximally along handle 530 to a second position on handle 530, to thereby retract retrieval end 519 of release knot 516, which pulls shunt 10 proximally via knot connection 518 within the lumen of sheath 502, as shown in FIG. 7J. As shunt 10 is pulled proximally within sheath 502, distal region 504 of sheath 502 applies a force against the deployed distal portion of shunt 10, which causes the distal portion to transition to its collapsed delivery state within the lumen of sheath 502. As described above, in some embodiments, lock 539 of retrieval actuator 538 may be required to transition from its locked configuration to its unlocked configuration prior to moving retrieval actuator 538 from the first position to the second position.

When shunt 10 is completely in its collapsed delivery state within sheath 502, device 500 may be removed from the patient, e.g., by moving handle 530 proximally, as shown in FIG. 7K. Alternatively, device 500 may be repositioned relative to atrial septum AS, such that shunt 10 may be implanted atrial septum AS in accordance with the delivery methods described above with regard to FIGS. 7E to 7H.

Alternatively, as described above, release knot 516 may remain coupled to shunt 10 during full deployment of shunt 10 at atrial septum AS. Accordingly, release actuator 536 may not be actuated prior to retracting device 500 proximally relative to atrial septum AS to thereby complete full deployment of the shunt by deploying the proximal portion of shunt 10 within the right atrium. In this embodiment, upon satisfactory full deployment of shunt 10 at atrial septum AS, release actuator 536 may then be actuated to disassemble knot connection 518 and disengage release knot 516 from shunt 10. If deployment is unsatisfactory, retrieval end 519 may be retracted proximally, e.g., via actuation of retrieval actuator 538, to facilitate transition of the proximal portion and/or the middle portion of shunt 10 toward the collapsed delivery state, as described above, such that shunt 10 may be retracted back into the lumen of sheath 502 for redeployment or removal.

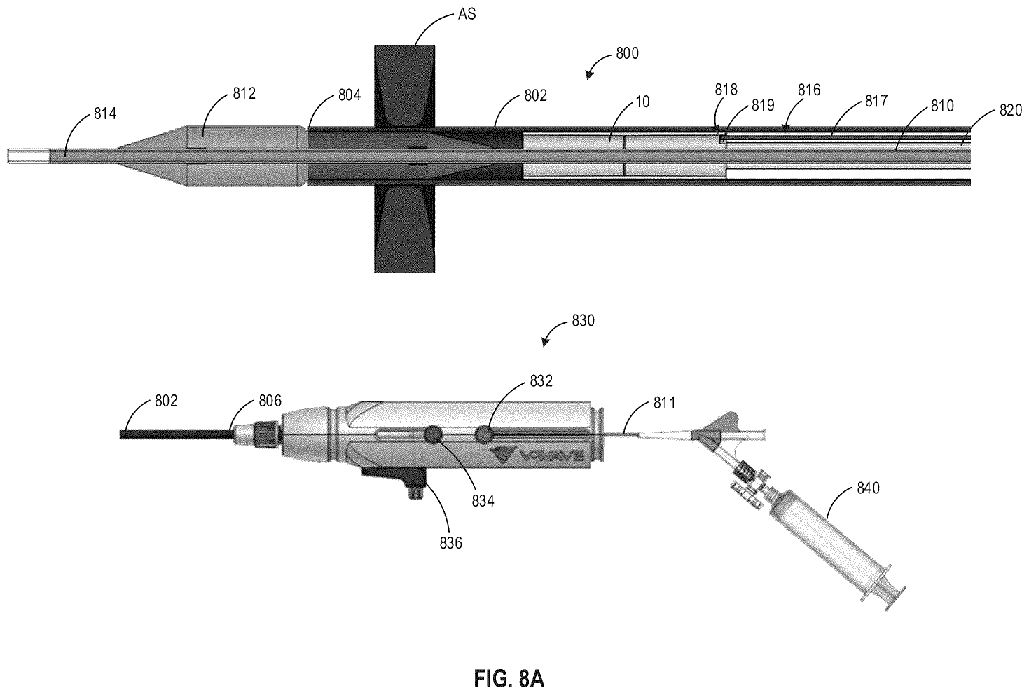

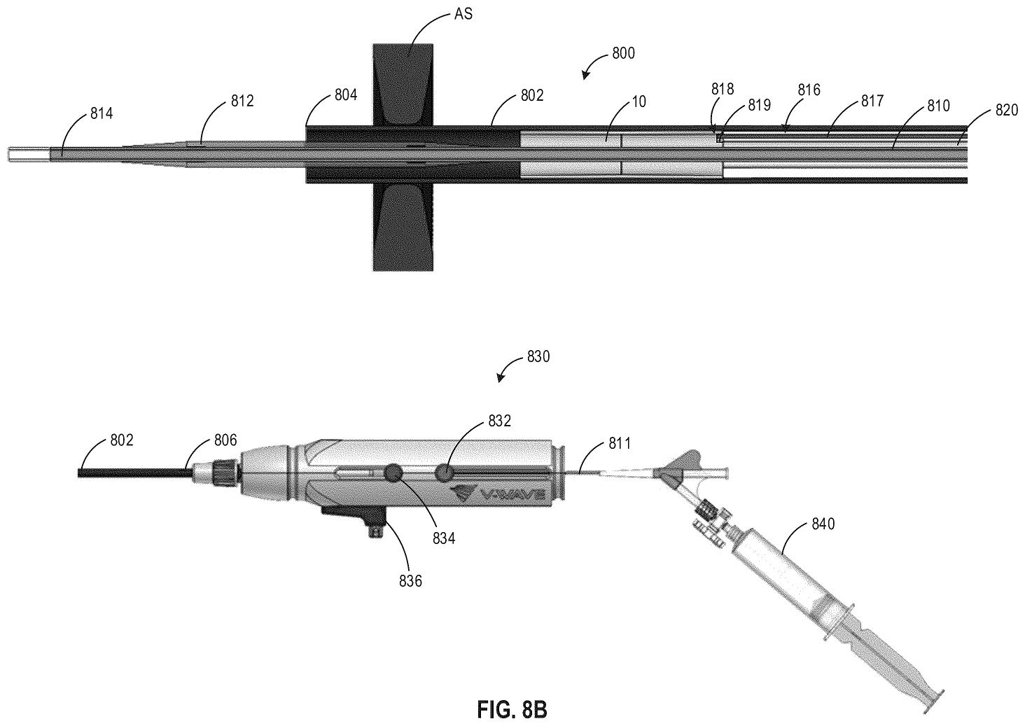

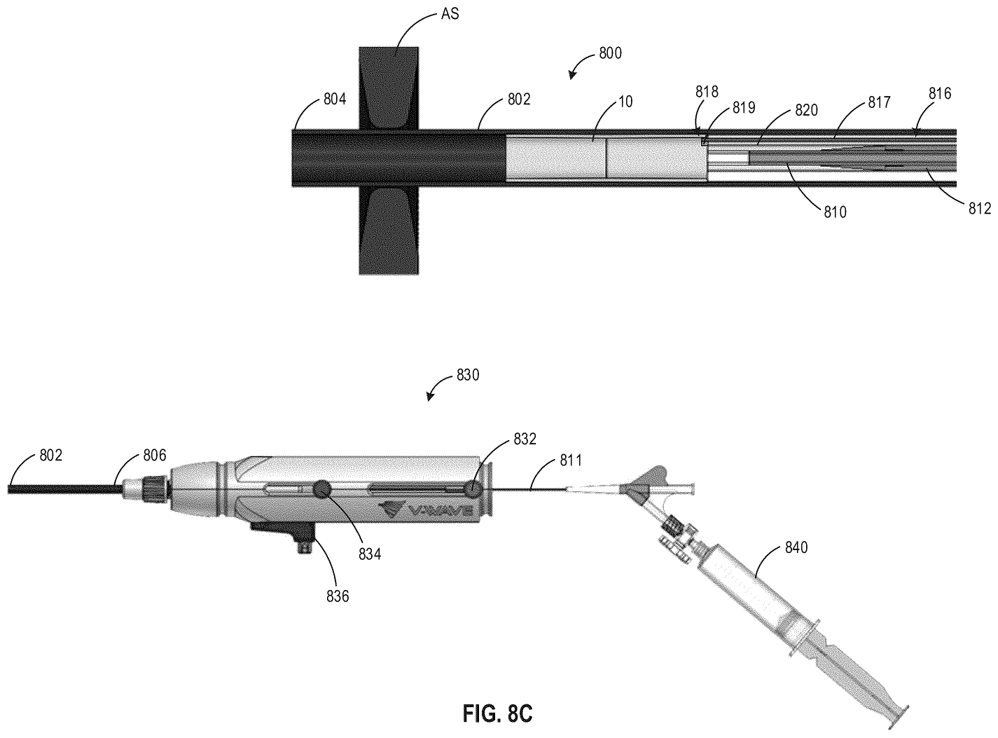

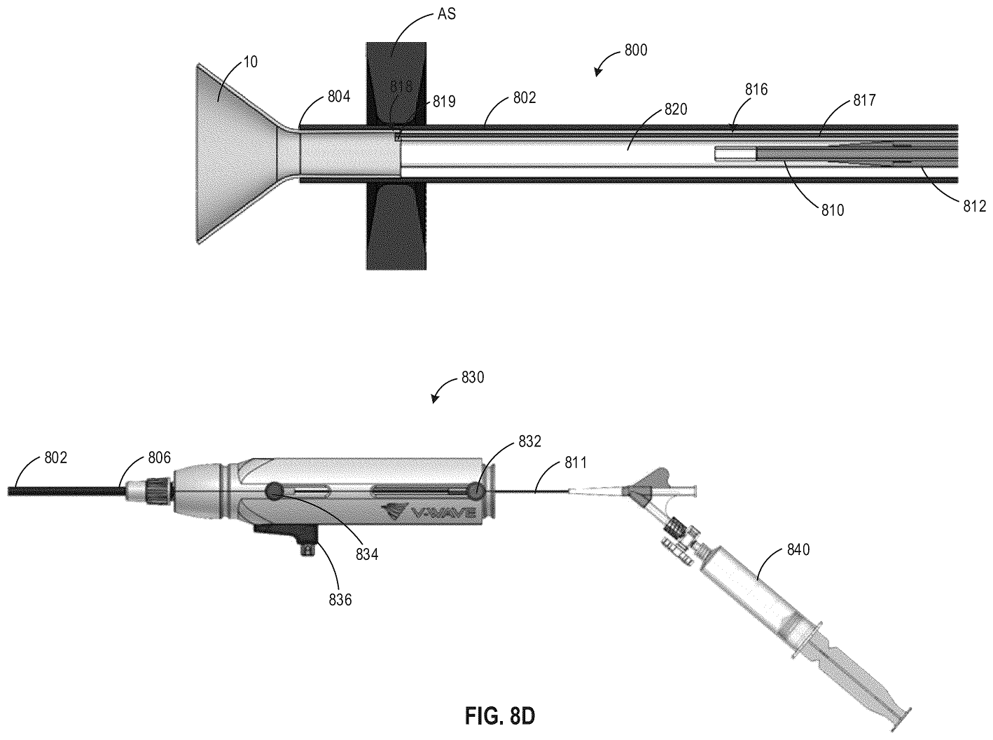

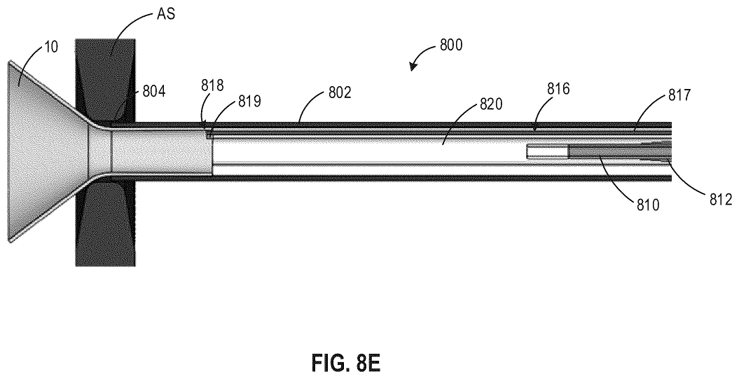

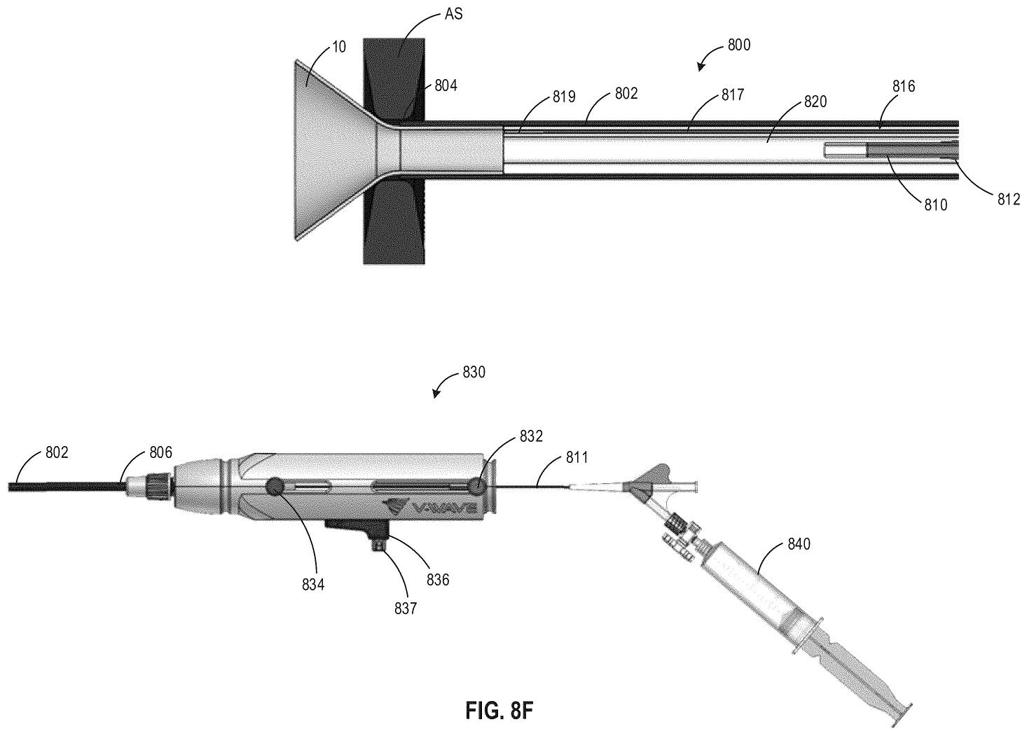

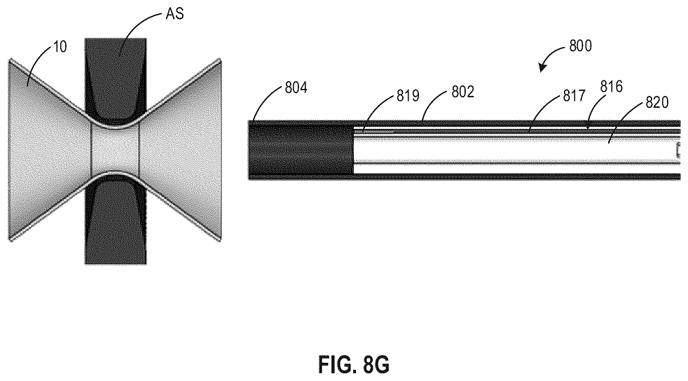

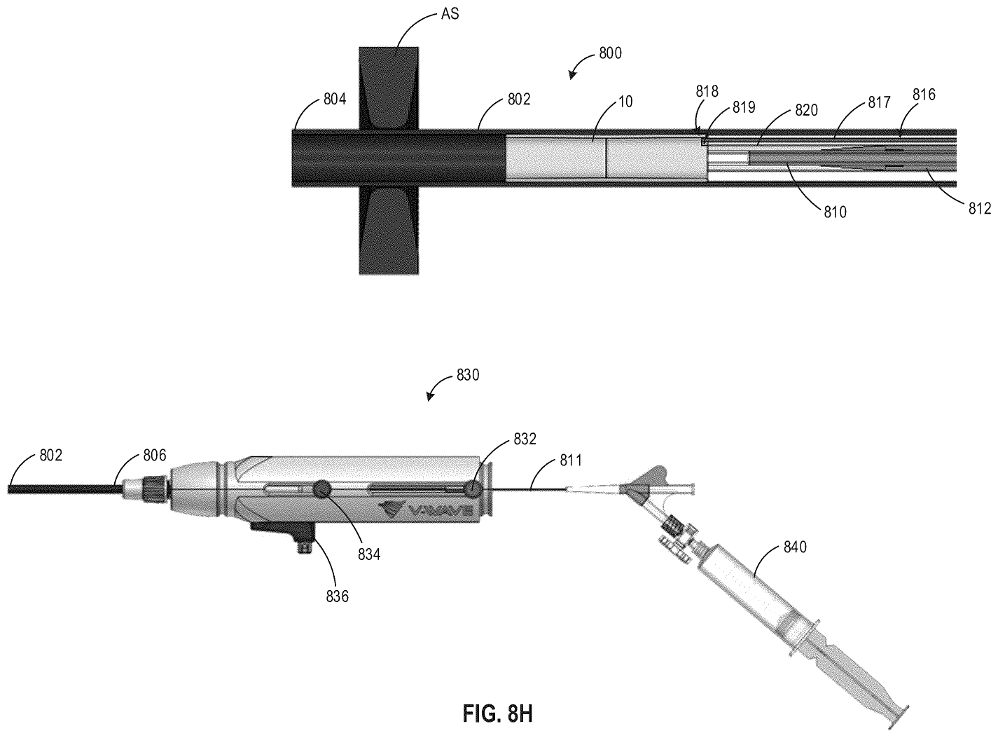

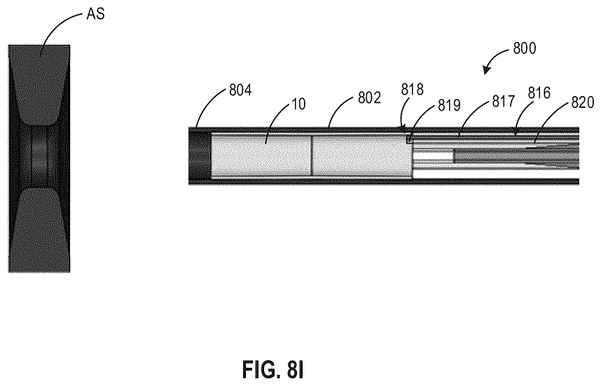

Referring now to FIGS. 8A to 8I, exemplary method steps for delivering interatrial shunt device 10 to the atrial septum via exemplary delivery device 800 operatively coupled to handle 830 are provided. Device 800 may be constructed similar to delivery device 500. For example, sheath 802 having distal region 804 and proximal region 806 corresponds to sheath 502 having distal region 504 and proximal region 506, balloon catheter 810 having inflatable balloon 812 and fluid lumen 811 corresponds to balloon catheter 510 having inflatable balloon 512 and fluid lumen 511, pusher 820 corresponds with pusher 520, and release knot 816 having release end 817, knot connection 818, and retrieval end 819 corresponds with release knot 516 having release end 517, knot connection 518, and retrieval end 519. Device 800 differs from delivery device 500 in that, rather than extending through the length of pusher 820 from knot connection 818 to a retrieval actuator of handle 830, retrieval end 819 may be coupled to the distal portion of pusher 820. Accordingly, handle 830, which may be constructed similar to handle 530 such that handle 830 is coupled to proximal region 806 of sheath 802, balloon catheter actuator 832 corresponds to balloon catheter actuator 532, pusher actuator 834 corresponds to pusher actuator 534, release actuator 836 corresponds to release actuator 536, and fluid source 840 corresponds to fluid source 540, does not need a separate retrieval actuator. For example, actuation of actuator 832, e.g., moving actuator 832 proximally along handle 830, causes movement of pusher 820, and accordingly shunt 10 and retrieval end 819 coupled thereto, proximally within the lumen of sheath 802. Moreover, pusher 820 does not have a separate lumen for receiving retrieval end 819 therethrough.

Like release end 517, as described above, release end 817 may have slack within device 800, such that actuation of release actuator 836 may not cause disassembly of knot connection 818 until shunt 10 is halfway deployed from sheath 802. Alternatively, in some embodiments, release actuator 836 may be releasably coupled to pusher actuator 834, such that release actuator 836 moves along with pusher actuator 834 when pusher actuator 834 is actuated to move pusher 820, and accordingly shunt 10, distally within sheath 802. Accordingly, release end 817 may not have slack within device 800 as release end 817 moves distally along with pusher 820 via movement of release actuator 836. Release actuator 836 may be decoupled from pusher actuator 834, e.g., via lock 837 or another locking mechanism coupling release actuator 836 and pusher actuator 834, and then independently actuated to pull release end 817 to disassemble knot connection 818 and disengage release knot 816 from shunt 10, as described above.

In some embodiments, release actuator 836 may include a rope clutch mechanism through which release end 817 may be passed through. For example, the rope clutch mechanism may be in an open state during actuation of pusher actuator 834, such that release end 817 moves through the rope clutch mechanism as pusher 820, and accordingly retrieval end 819 coupled thereto, are moved distally through sheath 802. If halfway retrieval of shunt 10 is desirable, as described above, pusher actuator 834 may be actuated to retract pusher 820, and accordingly shunt 10 via knot connection 818, within sheath 802. To disengage release knot 816 from shunt 10, the rope clutch mechanism may be transitioned to a closed state, e.g., via lock 837 or another closing mechanism operatively coupled to the rope clutch mechanism, to fix release end 817 to release actuator 836, such that actuation of release actuator 836 pulls on release end 817 to disassemble knot connection 818.

Like the method steps for delivery shunt 10 via device 500 described above with regard to FIGS. 7A-7H, as shown in FIG. 8A, device 800 with balloon 812 in an inflated expanded state adjacent distal region 804 of sheath 802 may be delivered through a hole in atrial septum AS, such that balloon 812 enlarges/dilates the puncture of atrial septum AS as the tissue surrounding the puncture is smoothly guided over the distal portion of balloon 812, followed by distal region 804 of sheath 802. Further, balloon 812 may be deflated, as shown in FIG. 8B, and retracted proximally within a lumen of pusher 820 via actuator 832, as shown in FIG. 8C. As shown in FIG. 8D, pusher 820 may be advanced distally within sheath 802 via actuator 834, such that the distal end of pusher 820 engages with the proximal end of shunt 10 and moves shunt 10 distally within sheath 802 until the distal portion of shunt 10 extends beyond distal region 804 and deploys within the left atrium.

As shown in FIG. 8E, device 800 may then be moved proximally, e.g., by moving handle 830 proximally, until the distal portion of shunt 10 contacts atrial septum AS. Next, release end 817 may be pulled proximally via release actuator 836 to disassemble knot connection 818 and disengage release knot 818 from shunt 10, while retrieval end 819 remains coupled to the distal portion of pusher 820, as shown in FIG. 8F. In some embodiments, lock 837 of release actuator 836 may be required to transition from its locked configuration to its unlocked configuration prior to actuating release actuator 836. As shown in FIG. 8G, device 800 may be retracted proximally while atrial septum AS maintains shunt 10 in place until the proximal portion of shunt 10 is exposed from sheath 802 and deploys within the right atrium. Delivery device 800 may then be removed from the patient, leaving shunt 10 implanted. As described above, balloon 81 may be re-inflated adjacent to distal region 804 prior to removal of device 800 from the patient.

As described above, after the distal portion of shunt 10 is deployed, e.g., within the left atrium as shown in FIG. 8D or in another improper location, it may be desirable to transition shunt 10 back to its collapsed delivery state within sheath 802 and retrieve shunt 10. Accordingly, as shown in FIG. 8H, actuator 832 may be actuated, e.g., moved proximally along handle 530, to thereby move pusher 820 proximally within the lumen of sheath 802, which pulls retrieval end 819 and shunt 10 proximally via knot connection 818 within the lumen of sheath 802 until the distal portion of shunt 10 transitions to its collapsed delivery state within sheath 802. Device 800 may then be removed from the patient, as shown in FIG. 8I, or alternatively, device 800 may be repositioned relative to atrial septum AS, such that shunt 10 may be implanted atrial septum AS in accordance with the delivery methods described above with regard to FIGS. 8D to 8G.

While various illustrative embodiments of the invention are described above, it will be apparent to one skilled in the art that various changes and modifications may be made therein without departing from the invention. The appended claims are intended to cover all such changes and modifications that fall within the true scope of the invention.

1. An apparatus for delivering a shunt to an atrial septum of a patient, the apparatus comprising:
a sheath having a proximal region, a distal region, and a sheath lumen extending therethrough, the sheath lumen sized and shaped to receive the shunt in a collapsed delivery state;
a balloon catheter configured to be moveably disposed within the sheath lumen, the balloon catheter comprising a balloon configured to transition between a deflated collapsed state and an inflated expanded state adjacent to the distal region to form a continuous, step-free transition between the balloon and the distal region of the sheath; and
a handle comprising one or more actuators configured to be actuated to deploy the shunt at the atrial septum.

2. The apparatus of claim 1, further comprising:
a pusher slidably disposed within the sheath lumen, the pusher operatively coupled to a pusher actuator of the one or more actuators of the handle,
wherein the pusher actuator is configured to be actuated to move the pusher within the sheath lumen.

3. The apparatus of claim 2, wherein the pusher actuator is configured to be actuated to move the pusher distally relative to the sheath, such that a distal end of the pusher engages with a proximal portion of the shunt in the collapsed delivery state to thereby move the shunt distally relative to the sheath until a distal portion of the shunt is exposed beyond the distal region of the sheath and transitions from the collapsed delivery state to an expanded deployed state.

4. The apparatus of claim 2, further comprising a release knot slidably disposed within the sheath lumen, the release knot configured to be releasably engaged with the shunt via a hitch knot.

5. The apparatus of claim 4, wherein a first end of the release knot is operatively coupled to a release actuator of the one or more actuators of the handle and a second end of the release knot is operatively coupled to a retrieval actuator of the one or more actuators of the handle, such that actuation of the release actuator causes the hitch knot to disassemble and disengage the release knot from the shunt, and actuation of the retrieval actuator causes retraction of the shunt proximally within the sheath lumen via the hitch knot for halfway retrieval of the shunt.

6. The apparatus of claim 4, wherein a first end of the release knot is operatively coupled to a release actuator of the one or more actuators of the handle and a second end of the release knot is operatively coupled to a distal portion of the pusher, such that actuation of the release actuator causes the hitch knot to disassemble and disengage the release knot from the shunt, and actuation of the pusher actuator causes retraction of the shunt proximally within the sheath lumen via the hitch knot for halfway retrieval of the shunt.

7. The apparatus of claim 4, wherein the hitch knot comprises a painters hitch knot or a Quick Tie and Release (QTaR) hitch knot.

8. The apparatus of claim 1, wherein the balloon is configured to be deflated to permit deployment of the shunt through the distal region of the sheath.

9. The apparatus of claim 1, wherein the balloon catheter comprises a fluid lumen configured to fluidically couple the balloon and a fluid source.

10. The apparatus of claim 1, wherein the balloon catheter is operatively coupled to a balloon catheter actuator of the one or more actuators of the handle, and wherein actuation of the balloon catheter actuator causes the balloon catheter to move relative to the sheath.

11. A method for delivering a shunt to an atrial septum of a patient, the method comprising:
inflating a balloon adjacent to a distal region of a sheath to form a continuous, step-free transition between the balloon and the distal region of the sheath, the balloon disposed on a distal portion of a balloon catheter slidably disposed within a lumen of the sheath;
delivering the inflated balloon and the sheath through an opening of the atrial septum, such that the inflated balloon and the sheath dilates the opening of the atrial septum;
deflating the balloon;
advancing the shunt distally within the lumen of the sheath in a collapsed delivery state until a distal portion of the shunt is exposed beyond the distal region of the sheath and transitions to an expanded deployed state within a first atrium;
retracting the sheath proximally relative to the atrial septum until a proximal portion of the shunt is exposed beyond the distal region of the sheath and transitions to the expanded deployed state within a second atrium, such that the shunt is deployed at the atrial septum; and
removing the sheath and the balloon catheter from the patient.

12. The method of claim 11, wherein the proximal portion of the shunt is releasably engaged with a release knot, such that pulling a first end of the release knot causes the release knot to disengage from the shunt, and pulling a second end of the release knot causes retraction of the shunt proximally within the lumen of the sheath for halfway retrieval of the shunt.

13. The method of claim 12, wherein the release knot is releasably engaged with the shunt via a painters hitch knot or a Quick Tie and Release (QTaR) hitch knot.

14. The method of claim 12, wherein, prior to retracting the sheath proximally relative to the atrial septum until the proximal portion of the shunt is exposed beyond the distal region of the sheath, the method further comprises pulling the first end of the release knot to disengage the release knot from the shunt.

15. The method of claim 12, wherein, prior to retracting the sheath proximally relative to the atrial septum until the proximal portion of the shunt is exposed beyond the distal region of the sheath, the method further comprises pulling the second end of the release knot to transition the distal portion of the shunt to the collapsed delivery state within the lumen of the sheath for halfway retrieval of the shunt.

16. The method of claim 11, wherein, prior to retracting the sheath proximally relative to the atrial septum until the proximal portion of the shunt is exposed beyond the distal region of the sheath, the method further comprises retracting the sheath and the shunt proximally relative to the atrial septum until the distal portion of the shunt contacts the atrial septum from within the first atrium.

17. The method of claim 11, wherein advancing the shunt distally within the lumen of the sheath comprises advancing a pusher distally within the lumen of the sheath, such that a distal end of the pusher engages with the proximal portion of the shunt in the collapses delivery state within the lumen of the sheath.

18. The method of claim 17, wherein, prior to retracting the sheath proximally relative to the atrial septum until the proximal portion of the shunt is exposed beyond the distal region of the sheath, the method further comprises:
retracting the shunt proximally within the lumen of the sheath to transition the distal portion of the shunt to the collapsed delivery state within the lumen of the sheath for halfway retrieval of the shunt.

19. (canceled)

20. The method of claim 11, further comprising retracting the balloon catheter and the deflated balloon proximally within the lumen of the sheath to a position proximal to the shunt in the collapsed delivery state prior to advancing the shunt within the lumen of the sheath.

21. An apparatus for delivering a shunt to an atrial septum of a patient, the apparatus comprising:
a sheath configured to be advanced through a hole in the atrial septum, the sheath having a proximal region, a distal region, and a sheath lumen extending therethrough, the sheath lumen sized and shaped to receive the shunt in a collapsed delivery state; and
a dilator moveably disposed within the sheath lumen, the dilator comprising an expandable portion configured to transition between a first state and a second state where the expandable portion engages with the distal region of the sheath,
wherein the dilator and the sheath are configured to dilate the hole in the atrial septum as tissue surrounding the hole is smoothly guided over the distal portion of the dilator and the sheath as the apparatus is advanced through hole in the atrial septum.

22. The apparatus of claim 21, wherein the dilator comprises a guidewire lumen sized and shaped to receive a guidewire.

23-49. (canceled)

50. The apparatus of claim 21, wherein the expandable portion of the dilator comprises a balloon coupled to a balloon catheter configured to be moveably disposed within the sheath lumen, the balloon configured to transition between the first state and the second state adjacent to the distal region to form a continuous, step-free transition between the balloon and the distal region of the sheath.

51. The apparatus of claim 50, further comprising a pusher slidably disposed within the sheath lumen, the pusher comprising a pusher lumen sized and shaped to slidably receive the balloon catheter therethrough, and a distal end configured to engage with a proximal portion of the shunt in the collapsed delivery state to thereby move the shunt distally relative to the sheath.

52. The apparatus of claim 50, further comprising a release knot slidably disposed within the sheath lumen, the release knot configured to be releasably engaged with the shunt via a hitch knot, such that pulling a first end of the release knot causes the release knot to disengage from the shunt, and pulling a second end of the release knot causes retraction of the shunt proximally within the sheath lumen for halfway retrieval of the shunt.

53. The apparatus of claim 52, wherein the hitch knot comprises a painters hitch knot or a Quick Tie and Release (QTaR) hitch knot.

54. The apparatus of claim 52, wherein the release knot is configured to be releasably engaged with a proximal portion of the shunt.

55. The apparatus of claim 54, wherein the first and second ends of the release knot pass through a central passageway of the shunt toward a middle portion of the shunt, and loop around an outer surface of the middle portion of the shunt and back toward the hitch knot, such that pulling the second end of the release knot causes the shunt to transition toward the collapsed delivery state.

56. The apparatus of claim 52, wherein the release knot is configured to be releasably engaged with a middle portion of the shunt, and wherein the first and second ends of the release knot loop around an outer surface of the middle portion of the shunt, such that pulling the second end of the release knot causes the shunt to transition toward the collapsed delivery state.

57. The apparatus of claim 50, wherein the balloon is configured to be deflated to permit deployment of the shunt through the distal region of the sheath.

58. The apparatus of claim 50, wherein the balloon catheter comprises a fluid lumen configured to fluidically couple the balloon and a fluid source.
